(19) United States
(12) Patent Application Publication
(10) Pub. No.: US 2016/0168573 A1
(43) Pub. Date: Jun. 16, 2016

(54) LIVER CANCER RELATED GENES-SPECIFIC siRNA, DOUBLE-STRANDED OLIGO RNA MOLECULES COMPRISING THE siRNA, AND COMPOSITION FOR PREVENTING OR TREATING CANCER COMPRISING THE SAME

(71) Applicants: BIONEER CORPORATION, Daejeon (KR); SANOFI-AVENTIS KOREA CO., LTD., Seoul (KR)

(72) Inventors: Jeiwook Chae, Daejeon (KR); Han Oh Park, Daejeon (KR); Pyoung Oh Yoon, Daejeon (KR); Boram Han, Daejeon (KR); Han-na Kim, Jeollabuk-do (KR); Sung-II Yun, Daejeon (KR); Jun Hong Park, Daejeon (KR); Youngho Ko, Seoul (KR); Gi-Eun Choi, Gyeonggi-do (KR); Junsoo Jung, Seoul (KR); Jae Eun Kim, Seoul (KR)

(21) Appl. No.: 14/902,808

(22) PCT Filed: Jul. 9, 2014
(86) PCT No.: PCT/KR2014/006145

§ 371 Date: Jan. 4, 2016

(30) Foreign Application Priority Data
Jul. 9, 2013 (KR) 10-2013-0080579

(51) Int. Cl. C12N 15/113

(52) U.S. Cl. CPC C12N 15/1135; C12N 2310/14

(57) ABSTRACT

There is provided a liver cancer related specific siRNA and high efficiency double-stranded oligo RNA molecules containing the same. The double-stranded oligo RNA molecules have a structure in which hydrophilic and hydrophobic compounds are conjugated to both ends of the double-stranded oligo RNA molecules by a simple covalent bond or a linker-mediated covalent bond in order to be efficiently delivered into cells and may be converted into nanoparticles in an aqueous solution by hydrophobic interactions of the double-stranded oligo RNA molecules. The siRNA contained in the double-stranded oligo RNA molecules may be liver cancer related genes, particularly Gankyrin or BMI-1 specific siRNA.

In addition, the present invention relates to a method of preparing the double-stranded oligo RNA molecules, and a pharmaceutical composition for preventing or treating cancer, particularly, liver cancer, containing the double-stranded oligo RNA molecules.

### TECHNICAL FIELD

The present invention relates to a liver cancer related specific siRNA and high efficiency double-stranded oligo RNA molecules containing the same. The double-stranded oligo RNA molecules have a structure in which hydrophilic and hydrophobic compounds are conjugated to both ends of the double-stranded RNA molecules by a simple covalent bond or a linker-mediated covalent bond in order to be efficiently delivered into cells and may be converted into nanoparticles in an aqueous solution by hydrophobic interactions of the double-stranded oligo RNA molecules. The siRNA contained in the double-stranded oligo RNA molecules may be preferably liver cancer related genes, particularly Gankyrin or BMI-1 specific siRNA.

In addition, the present invention relates to a method of preparing the double-stranded oligo RNA molecules, and a pharmaceutical composition for preventing or treating cancer, particularly, liver cancer, containing the double-stranded oligo RNA molecules.

### BACKGROUND ART

A technology of suppressing expression of genes is an important tool in developing a therapeutic agent for treating diseases and validating a target. Among these technologies, since roles of RNA interference (hereinafter, referred to as ‘RNAi’) was found, it was found that the RNAi acts on sequence-specific mRNA in various kinds of mammalian cells (Silence of the Transcripts: RNA Interference in Medicine, J. Mol. Med. 83: 764-773, 2005). When long-chain double-stranded RNA is delivered into cells, the delivered double stranded RNA is converted into a small interfering RNA (hereinafter, referred to as ‘siRNA’) processed into 21 to 23 base pairs (bp) by endonuclease called Dicer, wherein the siRNA is bound to a RNA-induced silencing complex (RISC), and then a guide (antisense) strand recognizes and degrades the target mRNA, such that the siRNA sequence-specifically inhibits expression of the target gene (Nucleic-Acid Therapeutics: Basic Principles and Recent Applications, Nature Reviews Drug Discovery 1: 503-514, 2002).

According to Bertrand et al., it was found that the siRNA has a more excellent effect of inhibiting expression of the mRNA in vivo and in vitro as compared to antisense oligonucleotide (ASO) on the same target genes (Comparison of Antisense Oligonucleotides and siRNAs in Cell Culture and in Vivo, Biochem. Biophys. Res. Commun., 296: 1000-1004, 2002). In addition, since action mechanism of the siRNA is that the siRNA is complementarily bound to the target mRNA to sequence-specifically control the expression of the target genes, the target to which the siRNA may be applied may be remarkably enlarged as compared to the existing antibody based drugs or small molecule drugs (Progress towards in Vivo Use of siRNAs, Molecular Therapy 13(4): 664-670, 2006).

In spite of excellent effects and various uses of the siRNA, in order to develop the siRNA as a therapeutic agent, the siRNA should be effectively delivered into a target cell by improving stability of the siRNA in vivo and cell delivery efficiency (Harnessing in vivo siRNA delivery for drug discovery and therapeutic development, Drug Discov. Today 11(1-2): 67-73, January 2006).

In order to solve the above-mentioned problem, research into a technology of modifying some nucleotides or a backbone of the siRNA for improving the stability in vivo so as to have resistance against nuclease or using a carrier such as a viral vector, liposome, nanoparticles, or the like, has been actively conducted.

In a delivery system using the viral vector such as adenovirus, retrovirus, or the like, transfection efficiency is high, but immunogenicity and oncogenicity are also high. On the other hand, a non-viral delivery system including nanoparticles has low cell delivery efficiency as compared to the viral delivery system but has advantages in that the non-viral delivery system may have high stability in vivo, be target-specific delivered, improve delivery efficiency through uptake or internalization of RNAi oligonucleotide contained therein into cell or tissues, or the like, and does not almost cause cytotoxicity and immune stimulation, such that currently, the non-viral delivery system has been evaluated as a potential delivery system as compared to the viral delivery system (Nonviral Delivery of Synthetic siRNA in vivo, J. Clin. Invest., 117(12): 3623-3632, Dec. 3, 2007).

Among the non-viral delivery systems, in a method of using nanocarrier, nanoparticles are formed by using various polymers such as liposome, a cationic polymer complex, and the like, and then iRNA is supported on these nanoparticles, that is, nanocarriers to thereby be delivered into the cell. In the method of using the nanocarrier, a polymeric nanoparticle, polymer micelle, lipoplex, and the like, are mainly used. Among them, the lipoplex is composed of cationic lipid and interacts with anionic lipid of endosome in the cell to destabilize the endosome, thereby serving to deliver the iRNA into the cell (Proc. Natl. Acad. Sci. 15; 93(21): 11493-8, 1996).

In addition, it was known that the efficiency of the siRNA in vivo may be increased by conjugating chemical compound, or the like, to an end site of a passenger (sense) strand of the siRNA to allow the siRNA to have improved pharmacokinetic features (Nature 11; 432(7014): 173-8, 2004). In this case, the stability of the siRNA may be changed according to the property of the chemical compound conjugated to the end of the sense (passenger) or antisense (guide) strand of the siRNA. For example, siRNA conjugated with a polymer compound such as polyethylene glycol (PEG) interacts with an anionic phosphoric acid group of the siRNA in a presence of cationic compound to form a complex, thereby obtaining a carrier comprising improved siRNA stability (J. Control Release 129(2): 107-16, 2008). Particularly, since micelles made of a polymer complex have significantly uniform distribution and a structure spontaneously formed while comprising significantly small sizes as compared to microsphere, nanoparticles, or the like, which is another system used as a drug delivery carrier, there are advantages in that quality of a product may be easily managed and reproducibility may be easily secured.

Further, in order to improve intracellular delivery efficiency of the siRNA, a technology for securing stability of the siRNA and implementing efficient cell membrane permeability using a siRNA conjugate obtained by conjugating a hydrophilic compound (for example, polyethylene glycol (PEG)), which is a biocompatible polymer, to the siRNA via a simple covalent bond or a linker-mediated covalent bond has been developed (Korean Patent Registration No. 883471). However, even though the siRNA is chemically modified and conjugated to polyethylene glycol (PEC), disadvantages such as low stability in vivo and difficulty in delivering the siRNA into a target organ still remains. In order to solve these disadvantages, double-stranded oligo RNA molecules in which hydrophilic and hydrophobic compounds are bound to oligonucleotide, particularly, double-stranded oligo RNA such as siRNA have been developed. The molecules form self-assembled nanoparticles (which is referred to as self-assembled micelle inhibitory RNA (SAMiRNA™)) by hydrophobic interaction of the hydrophobic compound (See Korean Patent Registration No. 1224828). A SAMiRNA™ technology has advantages in that homogenous nanoparticles comprising significantly small sizes as compared to the existing delivery technologies may be obtained.

Meanwhile, one in four Koreans die due to cancer (first cause of death), and in accordance with the development of a diagnostic method and a date collecting method, aging population, environmental changes, and the like, the number of patients die due to cancer is significantly increased every year. In addition, generation of cancer and death due to cancer has also been increased in the world, such that a technology of preventing, diagnosing, and treating cancer is a common and urgent task for people (Bio-Technology (BT) Trends Report, Current Development Trend of New Drug for Major Diseases, Biotechnology Policy Research Center, 2007, Edition No. 72).

Cancer is one of the diseases resulting in death to the largest number of people around the world, and the development of an innovative cancer therapeutic agent may decrease medical expenses consumed at the time of treating cancer and create high added-value. Therapy of cancer is divided into surgery, radiation therapy, chemotherapy, and biological therapy. Among them, chemotherapy is a therapeutic method of suppressing proliferation of cancer cells or killing the cancer cells using a small molecule drug. Since much of the toxicities expressed by an anticancer drug are shown in normal cells, the anticancer drug has toxicity at some degree. In addition, the anticancer drug has resistance in that the drug has an anticancer effect but loses the anticancer effect after the drug is used for a constant period. Therefore, development of an anticancer drug capable of selectively acting on cancer cells and not generating resistance has been urgently demanded (Current Status of Conquering Cancer. BioWave 6 (19), 2004). Recently, development of a new anticancer drug target molecular features of cancer by securing molecular genetic information on cancer has been conducted, and it was reported that drug resistance is not generated in anticancer drugs targeting a specific molecular target. Therefore, a therapeutic agent comprising excellent effects and reducing adverse effects as compared to the existing anticancer drug may be developed by developing a gene therapeutic agent targeting the specific molecular target which only cancer cells have.

After it was known that expression of genes may be specifically and efficiently inhibited using a RNA interference phenomenon, research into siRNAs targeting various genes has been conducted as a therapeutic drug for cancer. Examples of these genes may include oncogene, an anti-apoptotic molecule, telomerase, growth factor receptor gene, signaling molecule, and the like, the research is mainly conducted toward inhibiting expression of genes required for survival of cancer cells or inducing apotosis (RNA Interference in Cancer, Biomolecular Engineering 23: 17-34, 2006).

Gankyrin is a p28 gene product, which is a control complex of 26S proteosome, and called p28^{GANK}. In addition, Gankyrin is an oncoprotein as a cell cycle regulator regulating activities of retinoblastoma protein (pRb) and p53, which are tumor suppressor genes. When Gankyrin is over-expressed, phosphorylation of pRb is increased, and an activity of p16^{INK4a} is inhibited, such that cell division may be accelerated (Gene Therapy Strategies for Hepatocellular Carcinoma, Journal of Biomedical Science 13(4): 453-68, 2006). When Gankyrin is decreased, the phosphorylation of pRb is decreased, caspace-8,9-mediated apotosis is increased, and tumor growth suppression was observed in a hepatocellular carcinoma (HCC) animal model (Use of Adenovirus-Delivered siRNA to Target Oncoprotein p28GANK in Hepatocellular Carcinoma, Gastroenterology 128(7): 2029-41, 2005).

In addition, B cell specific Molonet murine leukemia virus Insertion site 1 (BMI-1) is a transcriptional repressor and serves to regulate hematopoietic stem cells and neural stem cells. An enzymatic activity of BMI-1 was not known, but BMI-1 is a key regulatory factor of polycomb repressive complex-1 (PRC1) regulating a structure of chromatin and transcription activities of p16(ink4a) and p14(Arf), which are tumor suppressor proteins (BMI1 as a Novel Target for Drug Discovery in Cancer, J. Cell Biochem., 112(10): 2729-41, 2011). In the case in which a BMI-1 signal is not present in a normal cell, cell cycle progresses, such that apotosis is suppressed and division progresses. It was confirmed that BMI-1 is over-expressed in various cancers, and it was observed that when expression of BMI-1 is suppressed, cell proliferation, colony formation, and migration were remarkably inhibited in vitro and in vivo (Effect of siRNA-Mediated Silencing of BMI-1 Gene Expression on HeLa Cells, Cancer Science 101(2): 379-386, 2010).

As described above, possibilities of Gankyrin and BMI-1 as targets for anti-cancer drug are known, but development of a siRNA therapeutic agent for Gankyrin and BMI-1 and a technology of delivering the siRNA therapeutic agent is still insignificant. Therefore, a need for the siRNA therapeutic agent capable of specifically and efficiently inhibiting expression of Gankyrin and BMI-1 and the technology of delivering the siRNA therapeutic agent is significant in the market.

### DISCLOSURE

### Technical Problem

An object of the present invention is to provide a new siRNA capable of specifically and highly efficiently inhibiting expression of Gankyrin or BMI-1, double-stranded oligo RNA molecules containing the same, and a method of preparing the double-stranded oligo RNA molecules.

Another object of the present invention is to provide a pharmaceutical composition for preventing or treating cancer, particularly, liver cancer, containing Gankyrin or BMI-1 specific-siRNA or double-stranded oligo RNA molecules containing the Gankyrin or BMI-1 specific siRNA as an active ingredient.

Still another object of the present invention is to provide a method of preventing or treating cancer using the Gankyrin or BMI-1 specific siRNA or the double-stranded oligo RNA molecules containing the Gankyrin or BMI-1 specific siRNA.

### Technical Solution

According to an aspect of the present invention, there is provided Gankyrin or BMI-1 specific siRNA, which is liver cancer related gene, comprising a first oligonucleotide, which is a sense strand comprising any one sequence selected from SEQ ID NOs. 1 to 200 and a second oligonucleotide, which is an antisense strand complementary thereto.

The term “Gankyrin specific siRNA(s)” or “BMI-1 specific siRNA(s)” of the present invention means an siRNA(s) which is specific for gene encoding Gankyrin or BMI-1 protein. In addition, as long as the siRNAs retain the specificity to Gankyrin or BMI-1, the siRNAs of the present invention also comprise sense or antisense strand having one or more nucleotide deletion, insertion or substitution in sense strand of SEQ ID NOs: 1 to 200 or antisense strand complementary to the SEQ ID NOs: 1 to 200.

The SEQ ID NOs. 1 to 100 indicates sequences of the sense strand of the Gankyrin specific siRNA, and the SEQ ID NOs. 101 to 200 indicates sequences of the sense strand of the BMI-1 specific siRNA.

Preferably, the siRNA according to the present invention may have a sense strand of the Gankyrin specific siRNA comprising a sequence of the SEQ ID NO. 1, 10, 13, 56, or 99 or a sense strand of the BMI-1 specific siRNA comprising a sequence of the SEQ ID NO. 102, 180, 197, 199, or 200,

more preferably, the sense strand of the Gankyrin specific siRNA comprising the sequence of the SEQ ID NO. 1, 10, or 99 or the sense strand of the BMI-1 specific siRNA comprising the sequence of the SEQ ID NO. 102, 199, or 200, and

most preferably, the sense strand of the Gankyrin specific siRNA comprising the sequence of the SEQ ID NO. 1 or the sense strand of the BMI-1 specific siRNA comprising the sequence of the SEQ ID NO. 102.

The sense strand or antisense strand of the siRNA according to the present invention may be composed of 19 to 31 nucleotides.

Since the Gankyrin or BMI-1 specific siRNA provided in the present invention has a base sequence designed so as to be complementarily bound to mRNA encoding a gene corresponding thereto, the Gankyrin or BMI-1 specific siRNA may effectively suppress the expression of the corresponding gene. In addition, the Gankyrin or BMI-1 specific siRNA may include an overhang, which is a structure comprising one or at least two unpaired nucleotides at a 3′-end of the siRNA,

and in order to improve the stability of the siRNA in vivo, the Gankyrin or BMI-1 specific siRNA may include various modifications for imparting resistance against nuclease and decreasing non-specific immune reactions. Describing modification of the first or second oligonucleotide configuring the siRNA, at least one modification selected from modification by substitution of —OH group with —CH₃ (methyl), —OCH₃ (methoxy), —NH₂, —F (fluorine), —O-2-methoxyethyl, —O-propyl, —O-2-methylthioethyl, —O-3-aminopropyl, —O-3-dimethylaminopropyl, —O—N-methylacetamido, or —O-dimethylamidooxyethyl at a 2′-carbon site of a sugar structure in at least one nucleotide; modification by substitution of oxygen in the sugar structure in the nucleotide with sulfur; modification of a nucleotide bond into a phosphorothioate bond, a boranophosphate bond, or a methyl phosphonate bond may be combined to thereby be used, and modification into a peptide nucleic acid (PNA) type, a locked nucleic acid (LNA) type, or a unlocked nucleic acid (UNA) type may be used (Ann. Rev. Med. 55, 61-65 2004; U.S. Pat. No. 5,660,985; U.S. Pat. No. 5,958,691; U.S. Pat. No. 6,531,584; U.S. Pat. No. 5,808,023; U.S. Pat. No. 6,326,358; U.S. Pat. No. 6,175,001; Bioorg. Med. Chem. Lett. 14:1139-1143, 2003; RNA, 9:1034-1048, 2003; Nucleic Acid Res. 31:589-595, 2003; Nucleic Acids Research, 38(17) 5761-5773, 2010; Nucleic Acids Research, 39(5) 1823-1832, 2011).

The Gankyrin or BMI-1 specific siRNA provided in the present invention may significantly inhibit expression of corresponding proteins in addition to inhibiting expression the corresponding gene. Further, since it was known that the siRNA may improve sensitivity of radiation therapy or chemotherapy, which is a therapeutic method typically combined with a cancer-specific RNAi used to treat cancer (The Potential RNAi-based Combination Therapeutics. Arch. Pharm. Res. 34(1): 1-2, 201), the Gankyrin specific siRNA or BMI-1 specific siRNA according to the present invention may be used together with the existing radiation therapy or chemotherapy.

Further, in the case in which the Gankyrin specific siRNA and the BMI-1 specific siRNA according to the present invention are simultaneously used, expression of the corresponding genes is simultaneously inhibited, such that growth of cancer cells may be remarkably inhibited.

According to another aspect of the present invention, there is provided a conjugate in which hydrophilic and hydrophobic compounds are conjugated to both ends of the siRNA in order to efficiently deliver the liver cancer related genes, particularly Gankyrin or BMI-1 specific siRNA into the body and improve stability.

In the case in which the hydrophilic and hydrophobic compounds are bound to the siRNA as described above, self assembled nanoparticles are formed by the hydrophobic interaction of the hydrophobic compound (See Korean Patent Registration No. 1224828). This conjugate has significantly excellent delivery efficiency into the body and excellent stability in vivo, and uniformity of particles sizes is excellent, such that quality control (QC) may be easy. Therefore, this conjugate may have advantages in that a preparing process as a drug is simple.

As a specific example, the double-stranded oligo RNA molecules containing Gankyrin or BMI-1 specific siRNA according to the present invention may preferably have a structure of the following Structural Formula (1).

A-X—R—Y—B Structural Formula (1)

In Structural Formula (1), A is a hydrophilic compound, B is a hydrophobic compound, X and Y each are independently a simple covalent bond or linker-mediated covalent bond, and R is Gankyrin or BMI-1 specific siRNA.

As long as the siRNAs retain the specificity to Gankyrin or BMI-1, the Gankyrin or BMI-1 specific siRNAs of the present invention also comprise antisense strand which is partially complementary (mismatch) to the Gankyrin or BMI-1 mRNA, as well as antisense strand perfectly complementary (perfect match) to the Gankyrin or BMI-1 mRNA.

The antisense or sense strand of the siRNA of the present invention may have at least 70%, preferably 80%, more preferably 90%, and most preferably 95% of sequence homology or complementarity to the Gankyrin or BMI-1 mRNA sequence.

The siRNA may be a double stranded duplex or single stranded polynucleotide including, but not limited to, antisense oligonucleotide or miRNA.

More preferably, the double-stranded oligo RNA molecules containing Gankyrin or BMI-1 specific siRNA according to the present invention may have a structure of the following Structural Formula (2).

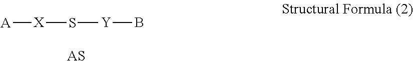

In Structural Formula (2), A, B, X, and Y have the same definitions as those in Structural Formula (1), respectively, S is a sense strand of the Gankyrin or BMI-1 specific siRNA, and AS is an antisense strand of the Gankyrin or BMI-1 specific siRNA.

More preferably, the double-stranded oligo RNA molecules containing Gankyrin or BMI-1 specific siRNA according to the present invention may have a structure of the following Structural Formula (3).

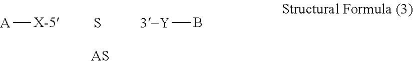

It will be apparent to those skilled in the art to which the present invention pertains that in Structural Formulas (1) to (3), one to three phosphate groups may be bound to a 5′-end of the antisense strand of the double-stranded oligo RNA molecules containing Gankyrin or BMI-1 specific siRNA and siRNA may be used instead of the siRNA.

The hydrophilic compound in Structural Formulas (1) to (3) may be preferably a cationic or non-ionic polymer compound comprising a molecular weight of 200 to 10,000, more preferably a non-ionic polymer compound comprising a molecular weight of 1,000 to 2,000. For example, as a hydrophilic polymer compound, a non-ionic hydrophilic polymer compound such as polyethylene glycol, polyvinyl pyrrolidone, polyoxazoline, and the like, may be preferably used, but the present invention is not limited thereto.

The hydrophobic compound B in Structural Formulas (1) to (3) may serve to form nanoparticles made of oligonucleotide molecules of Structural Formula (1) through the hydrophobic interaction. Preferably, the hydrophobic compound may have a molecular weight of 250 to 1,000, and a steroid derivative, a glyceride derivative, glycerol ether, polypropylene glycol, saturated or unsaturated C₁₂-C₅₀ hydrocarbon, diacyl phosphatidylcholine, fatty acid, phospholipid, lipopolyamine, or the like, may be used, but the present invention is not limited thereto. It may be apparent to those skilled in the art to which the present invention pertains that any hydrophobic compound may be used as long as the compound may satisfy the object of the present invention.

The steroid derivative may be selected from a group consisting of cholesterol, cholestanol, cholic acid, cholesteryl formate, cholestanyl formate, and cholesteryl amine, and the glyceride derivative may be selected from mono-, di-, and tri-glycerides, and the like. In this case, fatty acid of the glyceride may be preferably unsaturated or saturated C₁₂-C₅₀ fatty acid.

Particularly, among the hydrophobic compounds, the saturated or unsaturated hydrocarbon or cholesterol may be preferable in that they may be easily bound in a process of synthesizing the oligonucleotide molecules according to the present invention.

The hydrophobic compound may be bound to a distal end opposite to the hydrophilic compound and may be bound to any site of the sense or antisense strand of the siRNA.

The hydrophilic or hydrophobic compound in Structural Formulas (1) to (3) and the Gankyrin or BMI-1 specific siRNA according to the present invention may be bound to each other by a simple covalent bond or a linker-mediated covalent bond (X or Y). The linker mediating the covalent bond is covalently bound to the hydrophilic or hydrophobic compound at the end of the Gankyrin or BMI-1 specific siRNA, and as long as the linker may provide a degradable bond in a specific environment, as needed, the linker is not particularly limited. Therefore, as the linker, any compound bound in order to activate the Gankyrin or BMI-1 specific siRNA and/or the hydrophilic (or hydrophobic) compound in the process of preparing the double-stranded oligo RNA molecules according to the present invention may be used. The covalent bond may be any one of a non-degradable bond or a degradable bond. In this case, examples of the non-degradable bond may include an amide bond and a phosphate bond, and examples of the degradable bond may include a disulfide bond, an acid-degradable bond, an ester bond, an anhydride bond, a biodegradable bond, an enzyme-degradable bond, and the like, but the non-degradable or the degradable bond are not limited thereto.

In addition, as the Gankyrin or BMI-1 specific siRNA represented by R in Structural Formulas (1) to (3), any siRNA may be used without limitations as long as the siRNA may be specifically bound to Gankyrin or BMI-1. Preferably, in the present invention, the Gankyrin or BMI-1 specific siRNA is composed of the sense strand comprising any one sequence selected from the SEQ ID NOs. 1 to 200 and the antisense strand comprising a sequence complementary thereto.

The siRNA according to the present invention may have preferably the sense strand of the Gankyrin specific siRNA comprising the sequence of the SEQ ID NO. 1, 10, 13, 56, or or the sense strand of the BMI-1 specific siRNA comprising the sequence of the SEQ ID NO. 102, 180, 197, 199, or 200, more preferably, the sense strand of the Gankyrin specific siRNA comprising the sequence of the SEQ ID NO. 1, 10, or 99 or the sense strand of the BMI-1 specific siRNA comprising the sequence of the SEQ ID NO. 102, 199, or 200, and most preferably, the sense strand of the Gankyrin specific siRNA comprising the sequence of the SEQ ID NO. 1 or the sense strand of the BMI-1 specific siRNA comprising the sequence of the SEQ ID NO. 102.

Meanwhile, tumor tissue is significantly rigid and has diffusion-limitation as compared with normal tissue. Since this diffusion-limitation has a negative influence on movement of nutrients required for tumor growth, oxygen, waste materials such as carbon dioxide, the tumor tissue overcomes this diffusion-limitation by forming a blood vessel therearound through angiogenesis. The blood vessel generated through the angiogenesis in the tumor tissue may be a leaky and defective blood vessel comprising a leak of 100 nm to 2 um according to a kind of cancer. Therefore, the nanoparticles may easily pass through capillary endothelium of the cancer tissue comprising the leaky and defective structure as compared to organized capillary vessels of the normal tissue, such that the nanoparticles may easily approach the tumor interstitium during a circulation process in a blood vessels, and lymphatic drainage does not exist in the tumor tissue, such that drugs may be accumulated, which is called an ‘enhanced permeation and retention (EPR) effect’. Nanoparticles are tumor tissue-specifically delivered by this effect, which is referred to as ‘passive targeting’ (Nanoparticles for Drug Delivery in Cancer Treatment, Urol. Oncol., 26(1): 57-64, January-February, 2008). Active targeting means that a targeting moiety is bound to nanoparticles, and it was reported that the targeting moiety promotes preferential accumulation of the nanoparticles in the target tissue or improves internalization of the nanoparticles into the target cells (Does a Targeting Ligand Influence Nanoparticle Tumor Localization or Uptake Trends, Biotechnol. 26(10): 552-8m October, 2008, Epub. Aug. 21, 2008). In the active targeting, a target cell-specific material or a material, that is, the target moiety, capable of binding to over-expressed carbohydrate, receptor, or antigen is used (Nanotechnology in Cancer Therapeutics: Bioconjugated Nanoparticles for Drug Delivery, Mol. Cancer Ther., 5(8): 1909-1917, 2006).

Therefore, in the case in which the targeting moiety is provided in the double-stranded oligo RNA molecules containing Gankyrin or BMI-1 specific siRNA according to the present invention and the nanoparticles formed therefrom, delivery of the siRNA into the target cell may be efficiently promoted, such that the siRNA may be delivered into the target cell even at a relatively low concentration to thereby exhibit a high target gene expression regulatory function and prevent the Gankyrin or BMI-1 specific siRNA from being non-specifically delivered to other organs or cells.

Accordingly, the present invention provides double-stranded oligo RNA molecules in which a ligand L, particularly, a ligand specifically bound to a receptor promoting the internalization into the target cell through receptor-mediated endocytosis (RME) is additionally bound to the molecules represented by Structural Formulas (1) to (3), and a form in which the ligand is bound to the double-stranded RNA molecules represented by Structural Formula (1) has a structure of the following Structural Formula (4).

(Lᵢ-Zⱼ)-A-X—R—Y—B Structural Formula (4)

In Structural Formula (4), A, B, X, and Y have the same definitions as those in Structural Formulas (1) to (3), respectively, L is a ligand specifically bound to the receptor promoting the internalization into the target cell through receptor-mediated endocytosis (RME), and i and j each are independently 0 or 1.

Preferably, the ligand in Structural Formula (5) may be selected from a target receptor-specific antibody, aptamer, and peptide that have a receptor-mediated endocytic (REM) effect of target cell specifically promoting internalization; and chemicals, for example, folate (generally folate and folic acid are compatible with each other, and folate in the present invention means natural folate or active folate in the body), hexoamine such as N-acetyl galactosamine (NAG), sugars such as glucose, mannose, or the like, carbohydrate, or the like, but is not limited thereto.

According to still another aspect of the present invention, there is provided a method of preparing double-stranded oligo RNA molecules containing the Gankyrin or BMI-1 specific siRNA.

The method of preparing double-stranded oligo RNA molecules containing the Gankyrin or BMI-1 specific siRNA according to the present invention, for example, may include:

(1) binding a hydrophilic compound based on a solid support (the solid support used in the present invention is controlled pore glass (CPG);

(2) synthesizing a RNA single strand based on the solid support (CPG) to which the hydrophilic compound is bound;

(3) covalently binding a hydrophobic compound to a 5′-end of the RNA single strand;

(4) synthesizing a RNA single strand comprising a sequence complementary to that of the RNA single strand;

(5) separating RNA-polymer molecules and the RNA single strand from the solid support (CPG) after synthesizing is completed and then purifying the separated RNA-polymer molecules and RNA single strand; and

(6) preparing double-stranded oligo RNA molecules from the prepared RNA-polymer molecules and the RNA single strand comprising the complementary sequence through annealing.

When the preparation is completed after step (5), whether or not the desired RNA-polymer molecules and the RNA single strand are prepared may be confirmed by measuring molecular weights of the purified RNA-polymer molecules and the RNA single strand using a MALDI-TOF mass spectrometer. In the method, the synthesizing (step (4)) of the RNA single strand comprising the sequence complementary to that of the RNA single strand prepared in step (2) may be performed before step (1) or in any one step of step (1) to step (5).

In addition, the RNA single strand comprising the sequence complementary to that of the RNA single strand synthesized in step (2) may be used in a form in which a phosphate group is bound to the 5′-end.

Meanwhile, there is provided a method of preparing ligand bound-double stranded oligo RNA molecules in which a ligand is additionally bound to the double stranded oligo RNA molecules containing Gankyrin or BMI-1 specific siRNA according to the present invention.

The method of preparing the ligand bound-double-stranded oligo RNA molecules containing the Gankyrin or BMI-1 specific siRNA, for example, may include:

(1) binding a hydrophilic compound to a solid support (CPG) to which a functional group is bound;

(2) synthesizing a RNA single strand onto the solid support (CPG) to which the functional group-hydrophilic compound is bound;

(3) covalently binding a hydrophobic compound to a 5′-end of the RNA single strand;

(4) synthesizing a RNA single strand comprising a sequence complementary to that of the RNA single strand;

(5) separating functional group-RNA-polymer molecules and the RNA single strand comprising complementary sequence from the solid support (CPG) after synthesizing is completed;

(6) binding a ligand to an end of the hydrophilic compound using the functional group to prepare a ligand-RNA-polymer molecule single strand; and

(7) preparing ligand-double-stranded RNA-polymer molecules from the prepared ligand-RNA-polymer molecules and the RNA single strand comprising the complementary sequence through annealing.

When the preparation is completed after step (6), the ligand-RNA-polymer molecules and the RNA single strand comprising the complementary sequence are separated and purified. Then, whether or not the desired ligand-RNA-polymer molecules and the complementary RNA are prepared may be confirmed by measuring molecular weights of the purified RNA-polymer molecules and the RNA single strand using the MALDI-TOF mass spectrometer. The ligand-double-stranded oligo RNA-polymer molecules may be prepared from the prepared ligand-RNA-polymer molecules and the RNA single strand comprising the complementary sequence through annealing. In the method, the synthesizing (step (4)) of the RNA single strand comprising the sequence complementary to that of the RNA single strand prepared in step (3) may be performed as a independent synthetic process before step (1) or in any one step of step (1) to step (6).

According to still another aspect of the present invention, there is provided nanoparticles containing double-stranded oligo RNA molecules comprising Gankyrin and/or BMI-1 specific siRNA.

As described above, the double-stranded oligo RNA molecules comprising Gankyrin and/or BMI-1 specific siRNA are amphiphilic molecules containing both of the hydrophobic and hydrophilic compounds. A hydrophilic part may have affinity for water molecules existing in the body due to interaction such as a hydrogen bond with the water molecule, and the like, to thereby direct toward the outside, and the hydrophobic compounds may direct toward the inside due to the hydrophobic interaction therebetween, thereby forming thermally stable nanoparticles. That is, nanoparticles comprising a form in which the hydrophobic compound is positioned at the center of the nanoparticles and the hydrophilic compound is positioned in a direction toward the outside of the Gankyrin and/or BMI-1 specific siRNA to protect the Gankyrin and/or BMI-1 specific siRNA may be formed. The nanoparticles formed as described above may improve intracellular delivery efficiency of the Gankyrin and/or BMI-1 specific siRNA and effects of the siRNA.

The nanoparticles according to the present invention are characterized in that the nanoparticles are made of the double-stranded oligo RNA molecules comprising siRNAs comprising different sequences. Here, the siRNAs comprising different sequences may be different target genes, for example, Gankyrin or BMI-1 specific siRNA, or be siRNAs comprising different sequences while comprising specificity to the same target gene as each other.

In addition, double-stranded oligo RNA molecules containing another cancer-specific target specific siRNA except for the Gankyrin or BMI-1 specific siRNA may be contained in the nanoparticles according to the present invention.

According to still another aspect of the present invention, there is provided a composition for preventing or treating cancer containing: Gankyrin or BMI-1 specific siRNA; double-stranded oligo RNA molecules containing the same; and/or nanoparticles made of the double-stranded oligo RNA molecules.

The composition containing the Gankyrin or BMI-1 specific siRNA according to the present invention; the double-stranded oligo RNA molecules containing the same; and/or the nanoparticles made of the double-stranded oligo RNA molecules as active ingredients may induce proliferation and apoptosis of cancer cells to thereby exhibit effects of preventing or treating cancer. Therefore, the Gankyrin or BMI-1 specific siRNA according to the present invention and the composition containing the same may be effective in preventing or treating various cancers such as gastric cancer, lung cancer, pancreatic cancer, colon cancer, breast cancer, prostate cancer, ovarian cancer, and kidney cancer as well as liver cancer in which over-expression of the corresponding genes was reported.

Particularly, in the composition for preventing or treating cancer containing double-stranded oligo RNA molecules according to the present invention,

double-stranded oligo RNA molecules containing Gankyrin specific siRNA composed of a sense strand comprising any one sequence selected from SEQ ID NOs. 1 to 100, preferably, any one sequence selected from the SEQ ID NOs. 1, 10, 13, 56, and 99, more preferably, a sequence of the SEQ ID NOs. 1, 10, or 99, and most preferably, a sequence of the SEQ ID NO. 1 and an antisense strand comprising a sequence complementary to the sense strand, or

double-stranded oligo RNA molecules containing BMI-1 specific siRNA composed of a sense strand comprising any one sequence selected from SEQ ID NOs. 101 to 200, preferably, any one sequence selected from the SEQ ID NOs. 102, 180, 197, 199, and 200, more preferably, a sequence of SEQ ID NOs. 102, 199, or 200, and most preferably, a sequence of the SEQ ID NO. 102 and an antisense strand comprising a sequence complementary to the sense strand may be contained.

Alternatively, the double-stranded oligo RNA molecules containing Gankyrin specific siRNA and the double-stranded oligo RNA molecules containing BMI-1 specific siRNA may be included in a mixed form.

In addition, siRNA-specific to another cancer-specific target gene except for the Gankyrin or BMI-1 may be additionally contained in the composition of the present invention.

As described above, in the case of using the composition for preventing or treating cancer containing the double-stranded oligo RNA molecules containing Gankyrin specific siRNA and the BMI-1 specific siRNA, or containing the double-stranded oligo RNA molecules containing Gankyrin specific siRNA and another cancer-specific target specific siRNA in addition to the BMI-1 specific siRNA, a synergic effect may be obtained like a combination therapy commonly used to treat cancer.

The composition according to the present invention may prevent or treat, for example, liver cancer, gastric cancer, colon cancer, pancreatic cancer, prostate cancer, breast cancer, ovarian cancer, kidney cancer, lung cancer, and the like, but is not limited thereto.

In addition, the nanoparticles contained in the composition for preventing or treating cancer containing nanoparticles made of the double-stranded oligo RNA molecules according to the present invention may be purely composed of any one molecule selected from the double-stranded oligo RNA molecules containing the Gankyrin and BMI-1 specific siRNAs or composed of the double-stranded oligo RNA molecules containing the Gankyrin and BMI-1 specific siRNAs in a mixed form.

The composition according to the present invention may be prepared to further contain at least one kind of pharmaceutically acceptable carriers in addition to the active ingredients as describe above. The pharmaceutically acceptable carrier may be compatible with the active ingredients of the present invention, and any one of normal saline, sterile water, Ringer's solution, buffered saline, a dextrose solution, a maltodextrin solution, glycerol, and ethanol or a mixture of at least two thereof may be used. As needed, another general additive such as an antioxidant, a buffer solution, a bacteriostatic agent, or the like, may be added. In addition, the composition may be formulated into a formulation for injection such as an aqueous solution, a suspension, an emulsion, or the like, by additionally adding a diluent, a dispersant, a surfactant, a binder, and a lubricant.

Particularly, the composition may be preferably formulated into a lyophilized formulation.

A method generally known in the art to which the present invention pertains may be used in order to prepare the lyophilized formulation, and a stabilizer for lyophilization may be added. Further, the composition may be preferably formulated using an appropriate method known in the art or a method disclosed in Remington's pharmaceutical Science (Mack Publishing Company, Easton Pa.) according to the disease or the ingredient.

A content and an administration method of the active ingredient contained in the composition according to the present invention may be determined by a person comprising ordinary skill in the art based on patient's symptoms and severity of the disease. In addition, the composition may be formulated into various formulations such as powders, tablets, capsules, liquids, injections, ointments, syrups, and the like, and may be provided in a unit-dose container or multi-dose container, for example, a sealed ampoule, bottle, and the like.

The composition according to the present invention may be orally or parenterally administered. An administration route of the composition according to the present invention is not particularly limited, but oral, intravenous, intramuscular, intraarterial, intramedullary, intradural, intracardiac, transdermal, subcutaneous, abdominal, enteral, sublingual, or local administration may be performed. The dose of the composition according to the present invention may be various according to the weight, the age, the gender, the health status, and the diet of the patient, the administration time, the administration method, the excretion rate, the severity of the disease, or the like, and be easily determined by a person comprising ordinary skill in the art. In addition, the composition may be formulated into an appropriate formulation for clinical administration using a method known in the art.

According to another aspect of the present invention, there is provided a use of Gankyrin or BMI-1 specific siRNA, double-stranded oligo RNA molecules containing the same, and/or nanoparticles made of the double-stranded oligo RNA molecules in the manufacture of a medicament for preventing or treating cancer. According to still another aspect of the present invention, there is provided a method for preventing or treating cancer including administering the double-stranded oligo RNA molecules according to the present invention, nanoparticles including the double-stranded oligo RNA molecules, and the double-stranded oligo RNA molecules or the nanoparticles to a patient requiring treatment.

### Advantageous Effects

As set forth above, a composition for treating cancer containing Gankyrin and/or BMI-1 specific siRNA according to the present invention or double-stranded oligo RNA molecules containing the same may highly efficiently suppress expression of the Gankyrin and/or BMI-1 gene to effectively treat cancer, particularly, liver cancer without adverse effects, such that the composition may be significantly useful to treat the cancer in which there is no appropriate therapeutic agent.

### BRIEF DESCRIPTION OF DRAWINGS

FIG. 1 is a schematic diagram of a nanoparticle made of a double-stranded oligo RNA molecule according to the present invention;

FIG. 2 is a graph obtained by measuring sizes and polydispersity indexes (PDI) of nanoparticles made of double-stranded oligo RNA molecules comprising a sequence of SEQ ID NO. 1, 102, or 201 according to the present invention as a sense strand, where, SAMiRNA-Gank means nanoparticles made of double-stranded oligo RNA molecules comprising siRNA comprising a sequence of SEQ ID NO. 1 as a sense strand; SAMiRNA-BMI means nanoparticles made of double-stranded oligo RNA molecules comprising siRNA comprising a sequence of SEQ ID NO. 102 as a sense strand; and SAMiRNA-Gank+BMI means nanoparticles made of double-stranded oligo RNA molecules comprising siRNA comprising sense strand sequences of SEQ ID NOs. 1 and 102 as sense strands;

FIG. 3 is a graph of target gene expression inhibition levels confirmed after transfection with siRNAs (1 nM) comprising a sequence of SEQ ID NOs. 1 to 100 according to the present invention as a sense strand;

FIG. 4 is a graph of target gene expression inhibition levels confirmed after transfection with the siRNAs (0.2 nM) comprising the sequences of the SEQ ID NOs. 1 to 100 according to the present invention as a sense strand;

FIG. 5 is a graph of target gene expression inhibition levels confirmed after transfection with siRNAs (1 nM) comprising sequences of SEQ ID NOs. 101 to 200 according to the present invention as a sense strand;

FIG. 6 is a graph of target gene expression inhibition levels confirmed after transfection with the siRNAs (0.2 nM) comprising the sequence of the SEQ ID NOs. 101 to 200 according to the present invention as a sense strand;

FIGS. 7A and 7B are graphs obtained by confirming target gene expression inhibition levels after two kinds of liver cancer cells are treated with siRNAs comprising a sequence of SEQ ID NOs. 1, 10, 12, 35, 56, 61, 81, 88, and 99 according to the present invention as a sense strand, respectively, at a low concentration (A: Hep3B cell line, B: Huh-7 cell line);

FIGS. 8A and 8B are graphs obtained by confirming target gene expression inhibition levels after two kinds of liver cancer cells are treated with siRNAs comprising a sequence of SEQ ID NOs. 102, 124, 125, 180, 183, 193, 197, 198, 199, and 200 according to the present invention as a sense strand, respectively, at a low concentration (A: Hep3B cell line, B: Huh-7 cell line);

FIGS. 9A and 9B are graphs obtained by confirming inhibition concentrations 50% (IC50s) of siRNAs comprising sequences of the SEQ ID NOs. 1 and 102 according to the present invention as sense strands (A: IC50 of siRNA of SEQ ID NO. 1, B: IC50 of siRNA of SEQ ID NO. 102);

FIGS. 10A and 10B are photographs showing colony formation inhibition by corresponding siRNAs through colony forming assay (CFA) after two kinds of cancer cells are transfected with siRNAs of SEQ ID NOs. 1, 102, and 201 according to the present invention as a sense strand (A: colony forming assay using siRNA of SEQ ID NO. 1, B: colony forming assay using the siRNA of SEQ ID NO. 102);

FIG. 11 is a graph showing target gene expression inhibition at the time of co-transfection with siRNAs of the SEQ ID NOs. 1, 102, and 201 as a sense strand according to the present invention;

FIG. 12 is a graph showing cell viability reduction at the time of co-transfection with siRNAs of the SEQ ID NOs. 1, 102, and 201 according to the present invention as a sense strand;

FIG. 13 is a graph showing target gene expression inhibition at the time of intravenous injection of nanoparticles containing double-stranded RNA molecules comprising siRNA comprising sequences of SEQ ID NOs. 1 and 102 as sense strands in a liver cancer model, and the results shown in FIG. 13 are obtained by measuring a level of mRNA of the target gene expressed in an experimental group in which the nanoparticles containing the siRNA of SEQ ID NO. 102 are administered, as compared to a control group in which the nanoparticles containing the siRNA of SEQ ID NO. 201 are administered in tumor tissue after 48 hours of the last injection, and each number shown in X-axis indicates an individual; and

FIG. 14 is a graph showing a liver cancer growth suppression effect caused by intravenous injection of nanoparticles containing double-stranded RNA molecules comprising siRNA comprising sequences of SEQ ID NOs. 1, 102, and 201 according to the present invention into liver cancer models, wherein a liver cancer growth suppression level due to injection of the nanoparticles is confirmed by α-fetoprotein (AFP) value in serum, where, DPBS means a control group in which only Dulbecco's Phosphate-Buffered Saline (DPBS, used as a solvent) is injected; 201 means a control group in which the nanoparticles containing the siRNA of SEQ ID NO. 201 are injected at 5 mg/kg body weight; 1 means an experimental group in which the nanoparticles containing the siRNA of SEQ ID NO. 1 are injected at 5 mg/kg body weight; 102 means an experimental group in which the nanoparticles containing the siRNA of SEQ ID NO. 102 are injected at 5 mg/kg body weight; 1+102 means an experimental group in which the nanoparticles containing the double-stranded oligo RNA molecules containing the siRNA of SEQ ID NO. 1 and the double-stranded oligo RNA molecules comprising the siRNA of SEQ ID NO. 102 at the same content are injected at 5 mg/kg body weight (each of the double-stranded oligo RNA molecules are injected at 2.5 mg/kg body weight); and sorapenib means a positive control group.

### DETAILED DESCRIPTION OF THE EMBODIMENTS

Hereinafter, the present invention will be described in detail through Examples. However, these Examples are only to illustrate the present invention, and those skilled in the art will appreciate that these Examples are not to be construed as limiting a scope of the present invention.

### Example 1

### Design of Target Sequences of Gankyrin and BMI-1 Gene, and Preparation of siRNA

100 kinds of target sequences (sense strand) capable of binding to an mRNA sequence (NM_002814) of Gankyrin gene or an mRNA sequence (NM_005180) of BMI-1 gene were designed per each gene, and antisense siRNA strands comprising a sequence complementary to the desired base sequence were prepared. First, the desired base sequence to which the siRNA may bind was designed from the mRNA sequences of the corresponding genes using a Turbo si-Designer developed by Bioneer. siRNA for liver cancer related genes of the present invention has a double-stranded structure composed of a sense strand comprising 19 nucleotides and an antisense strand complementary thereto. In addition, siCONT (SEQ ID NO. 201), which is an siRNA comprising a sequence that does not inhibit expression of genes, was prepared. The siRNA is prepared by connecting a phosphodiester bond configuring a RNA backbone structure using β-cyanoethyl phosphoramidite (Nucleic Acids Research, 12: 4539-4557, 1984). More specifically, a reactant containing RNA comprising a desired length was obtained by repeating a series of processes consisting of deblocking, coupling, oxidation, and capping on a solid support on which nucleotides were adhered using an RNA synthesizer (384 Synthesizer, Bioneer, Korea). The RNA was separated from the reactant and purified using a HPLC (LC918, Japan Analytical Industry, Japan) equipped with a Daisogel C18 (Daiso, Japan) column. Then, whether or not the purified RNA coincides with the desired base sequence was confirmed using a MALDI-TOF mass spectrometer (Shimadzu, Japan). Next, the desired double-stranded siRNAs comprising sense strand of SEQ ID NOs. 1 to 201 were prepared by binding the sense and antisense RNA stands to each other (See Table 1).

TABLE 1
siRNA sense strand sequence
of the present invention

| SEQ | Target | |
|---|---|---|
| ID No. | Gene | Sequence |
| 1 | Gankyrin | CUGUUGAGAUUGUUCUACU |
| 2 | Gankyrin | CUGUACUCCCUUACAUUAU |
| 3 | Gankyrin | CGGCUGUUUUGACUGGCGU |
| 4 | Gankyrin | GGCCGGGAUGAGAUUGUAA |
| 5 | Gankyrin | GUGUCUAACCUAAUGGUCU |
| 6 | Gankyrin | GUGGCCUGGGUUUAAUACU |
| 7 | Gankyrin | CGCUGUCAUGUUACUGGAA |
| 8 | Gankyrin | CGCGCGACAAGUAGUUGCU |
| 9 | Gankyrin | GCUGGGACAGCGAAAUGGA |
| 10 | Gankyrin | GUUACUUGUUCGAAGCUUA |
| 11 | Gankyrin | GAAACAGAACAGCUCCAAU |
| 12 | Gankyrin | CCUUCUGGGAAAAGGUGCU |
| 13 | Gankyrin | CCAGAUGUUUCUAUGUGGA |
| 14 | Gankyrin | CCGGGAUGAGAUUGUAAAA |
| 15 | Gankyrin | GAGAGUGGAAGAAGCAAAA |
| 16 | Gankyrin | AGCCCUUCUGGGAAAAGGU |
| 17 | Gankyrin | AGGUGCUCAAGUGAAUGCU |
| 18 | Gankyrin | AGUUGGAUGGUGUGCUCUA |
| 19 | Gankyrin | GUUAAACAGCUUGGAUUUA |
| 20 | Gankyrin | GAGAGUAUUCUGGCCGAUA |
| 21 | Gankyrin | GUGGAAGGUUAAACAGCUU |
| 22 | Gankyrin | CUAAUUCUGUGGCUGUUGU |
| 23 | Gankyrin | GAGUAUUCUGGCCGAUAAA |
| 24 | Gankyrin | CCCAAGGAGCAAGUAUUUA |
| 25 | Gankyrin | CCCUCCCAUGUACCUUAUA |
| 26 | Gankyrin | GGAUGGUGUGCUCUAAAAU |
| 27 | Gankyrin | UUCUGCCAGAUGUUUCUAU |
| 28 | Gankyrin | GCUCGCGCGACAAGUAGUU |
| 29 | Gankyrin | GGUGUGUGUCUAACCUAAU |
| 30 | Gankyrin | UGGAUUCUGUAAUGUUCCU |
| 31 | Gankyrin | GAAUGAUAAAGACGAUGCA |
| 32 | Gankyrin | GCUCAAGUGAAUGCUGUCA |
| 33 | Gankyrin | CGAAAAACAGGCAUGAGAU |
| 34 | Gankyrin | GAGAUUGUUCUACUGUUGU |
| 35 | Gankyrin | GCAACAAGCUAGUUGUUCU |
| 36 | Gankyrin | CUGUCAUGUUACUGGAAGG |
| 37 | Gankyrin | AGAUGCUAAGGACCAUUAU |
| 38 | Gankyrin | CAGGUUGGUCUCCUCUUCA |
| 39 | Gankyrin | GCUUACAGCUUGUUUUCCA |
| 40 | Gankyrin | CUGAGUUACUUGUUCGAAG |
| 41 | Gankyrin | ACUUGGAGUGCCAGUGAAU |
| 42 | Gankyrin | AGCCCAUAUACCUAUGUAU |
| 43 | Gankyrin | CCAUUAUGAGGCUACAGCA |
| 44 | Gankyrin | GGUGGAAGGUUAAACAGCU |
| 45 | Gankyrin | CAUCUAUGAAUGAUGAAGU |
| 46 | Gankyrin | GUGUCCUACAAACUAAUGU |
| 47 | Gankyrin | GUGCACAAGACAUCAUCUA |
| 48 | Gankyrin | GUUCUACUGUUGUCGUAUA |
| 49 | Gankyrin | GUUGGAUGGUGUGCUCUAA |
| 50 | Gankyrin | CAGGACAGCAGAACUGCAU |
| 51 | Gankyrin | AACAAUAGCCCAUAUACCU |
| 52 | Gankyrin | CAGGCCUACGCCAAACGUU |
| 53 | Gankyrin | CUGGGUUUAAUACUCAAGA |
| 54 | Gankyrin | CGAAGCUUACAGCUUGUUU |
| 55 | Gankyrin | GUGUCAUCCUGUAUUGAAA |
| 56 | Gankyrin | AGACGAUGCAGGUUGGUCU |
| 57 | Gankyrin | UUCUAUGUGGAUUCUGUAA |
| 58 | Gankyrin | CUCCAAUAGCAACAAGCUA |
| 59 | Gankyrin | GCUACUAGAACUGACCAGG |
| 60 | Gankyrin | CCUCCCAUGUACCUUAUAU |
| 61 | Gankyrin | GUUCCUCCAUACAGUUAAA |
| 62 | Gankyrin | GUGUGUGUCUAACCUAAUG |
| 63 | Gankyrin | UGUAAUGUUCCUCCAUACA |
| 64 | Gankyrin | GUCCUACAAACUAAUGUAU |
| 65 | Gankyrin | GAAUAACUGUUGAGAUUGU |
| 66 | Gankyrin | GUUUUUGAUGGGUUGUUUA |
| 67 | Gankyrin | CAGUGAAUGAUAAAGACGA |
| 68 | Gankyrin | GUAUUCUGGCCGAUAAAUC |
| 69 | Gankyrin | GCUCUAACGGCUGUUUUGA |
| 70 | Gankyrin | GUUGCUGGGACAGCGAAAU |
| 71 | Gankyrin | GAUAAAUCCCUGGCUACUA |
| 72 | Gankyrin | GCCGGGAUGAGAUUGUAAA |
| 73 | Gankyrin | GGCUGUACUCCCUUACAUU |
| 74 | Gankyrin | GUCCCAAGGAGCAAGUAUU |
| 75 | Gankyrin | GAGAAUGGUGGAAGGUUAA |
| 76 | Gankyrin | GGUUAAACAGCUUGGAUUU |
| 77 | Gankyrin | CCCAGUGUCCUACAAACUA |
| 78 | Gankyrin | CCAGUGUCCUACAAACUAA |
| 79 | Gankyrin | CCUCCAUACAGUUAAAACA |
| 80 | Gankyrin | CGCCAAACGUUUCUGUUUU |
| 81 | Gankyrin | ACCUAAUGGUCUGCAACCU |
| 82 | Gankyrin | UCAAGAGAAUGGUGGAAGG |
| 83 | Gankyrin | AUCCAGAUGCUAAGGACCA |
| 84 | Gankyrin | CACUCAGGCCUACGCCAAA |
| 85 | Gankyrin | UCACUCAGGCCUACGCCAA |
| 86 | Gankyrin | CUGUUGUCGUAUAUUCUUC |
| 87 | Gankyrin | GGGUGUGUGUCUAACCUAA |
| 88 | Gankyrin | CGAUAAAUCCCUGGCUACU |
| 89 | Gankyrin | GUCAAUCAAAAUGGCUGUA |
| 90 | Gankyrin | GGCAUGAGAUCGCUGUCAU |
| 91 | Gankyrin | GGGCUAAUCCAGAUGCUAA |
| 92 | Gankyrin | CCAGAUGCUAAGGACCAUU |
| 93 | Gankyrin | GCCUGGGUUUAAUACUCAA |
| 94 | Gankyrin | GGGUUUAAUACUCAAGAGA |
| 95 | Gankyrin | CCCUCUCUGAAACAGAACA |
| 96 | Gankyrin | CCAAUAGCAACAAGCUAGU |
| 97 | Gankyrin | GUAUGUUGUGUUGUUGUCC |
| 98 | Gankyrin | CGAUGCAGGUUGGUCUCCU |
| 99 | Gankyrin | AGGAAGUUUUAAAGUACCU |
| 100 | Gankyrin | GGCUGUUUUGACUGGCGUA |
| 101 | BMI1 | GUGUGUUCAUCACCCAUCA |
| 102 | BMI1 | GAAAGUUUCUCAGAAGUAA |
| 103 | BMI1 | UGUCUACAUUCCUUCUGUA |
| 104 | BMI1 | GAAUUCUUUGACCAGAACA |
| 105 | BMI1 | CAUUGAUGCCACAACCAUA |
| 106 | BMI1 | GAAAUUCAACCAACGGAAA |
| 107 | BMI1 | CACAAGACCAGACCACUAC |
| 108 | BMI1 | CAGAGAGAUGGACUGACAA |
| 109 | BMI1 | GGGUACUUCAUUGAUGCCA |
| 110 | BMI1 | CCAGACCACUACUGAAUAU |
| 111 | BMI1 | GAGCUUCUACAGGUAUUUU |
| 112 | BMI1 | GUCUACAUUCCUUCUGUAA |
| 113 | BMI1 | CCUGGAGACCAGCAAGUAU |
| 114 | BMI1 | CUUUUUCUCUGUGUUAGGA |
| 115 | BMI1 | GUCACUGUGAAUAACGAUU |
| 116 | BMI1 | GUCGAACUUGGUGUGUGUU |
| 117 | BMI1 | CAGAGUUCGACCUACUUGU |
| 118 | BMI1 | GACUGACAAAUGCUGGAGA |
| 119 | BMI1 | CCAGAUUGAUGUCAUGUAU |
| 120 | BMI1 | CUCCAAGAUAUUGUAUACA |
| 121 | BMI1 | CAGGGCUUUUCAAAAAUGA |
| 122 | BMI1 | GUUAUUUGUGAGGGUGUUU |
| 123 | BMI1 | CUGGUUGAUACCUGAGACU |
| 124 | BMI1 | CGAGAAUCAAGAUCACUGA |
| 125 | BMI1 | CCACUACUGAAUAUAAGGU |
| 126 | BMI1 | GUCAGAUAAAACUCUCCAA |
| 127 | BMI1 | CCAACGGAAAGAAUAUGCA |
| 128 | BMI1 | CAACCAACGGAAAGAAUAU |
| 129 | BMI1 | GGUCAGAUAAAACUCUCCA |
| 130 | BMI1 | GACAUAAGCAUUGGGCCAU |
| 131 | BMI1 | GUACUCUGCAGUGGACAUA |
| 132 | BMI1 | GAGCAAGCAUGUUGAAUUU |
| 133 | BMI1 | GCUUGGCUCGCAUUCAUUU |
| 134 | BMI1 | GACUGUGAUGCACUUAAGA |
| 135 | BMI1 | GGUCCACUUCCAUUGAAAU |
| 136 | BMI1 | CGACCUACUUGUAAAAGAA |
| 137 | BMI1 | CUCACAUUUCCAGUACUAU |
| 138 | BMI1 | CCAGCAGGUUGCUAAAAGA |
| 139 | BMI1 | ACAAGACCAGACCACUACU |
| 140 | BMI1 | ACAUGUGACUAUCGUCCAA |
| 141 | BMI1 | AGUACUCUGCAGUGGACAU |
| 142 | BMI1 | GUGGUAUAGCAGUAAUUUU |
| 143 | BMI1 | GAGAAGGAAUGGUCCACUU |
| 144 | BMI1 | CUGUAGAAAACAAGUGCUU |
| 145 | BMI1 | GUAAGAAUCAGAUGGCAUU |
| 146 | BMI1 | GCCAAUAGACCUCGAAAAU |
| 147 | BMI1 | CGGGUACUACCGUUUAUUU |
| 148 | BMI1 | GGUGGUAUAGCAGUAAUUU |
| 149 | BMI1 | UAGAGCAAGCAUGUUGAAU |
| 150 | BMI1 | CAUUAUGCUUGUUGUACAA |
| 151 | BMI1 | CACCAAUCUUCUUUUGCCA |
| 152 | BMI1 | GUGUGUGUUCAUCACCCAU |
| 153 | BMI1 | GCCACAACCAUAAUAGAAU |
| 154 | BMI1 | CAGCAAGUAUUGUCCUAUU |
| 155 | BMI1 | GUAUGAGGAGGAACCUUUA |
| 156 | BMI1 | CCUCGAAAAUCAUCAGUAA |
| 157 | BMI1 | GGUUCGACCUUUGCAGAUA |
| 158 | BMI1 | GCAAUUGGCACAUCUUUCU |
| 159 | BMI1 | CCCAUUGUAAGUGUUGUUU |
| 160 | BMI1 | UCUAUGUAGCCAUGUCACU |
| 161 | BMI1 | UGCUUUGGUCGAACUUGGU |
| 162 | BMI1 | CACAACCAUAAUAGAAUGU |
| 163 | BMI1 | CUGUGAAUAACGAUUUCUU |
| 164 | BMI1 | GUAUUGUCCUAUUUGUGAU |
| 165 | BMI1 | CUGCAGCUCGCUUCAAGAU |
| 166 | BMI1 | CAGAUUGGAUCGGAAAGUA |
| 167 | BMI1 | CAGCGGUAACCACCAAUCU |
| 168 | BMI1 | CUGACAAAUGCUGGAGAAC |
| 169 | BMI1 | CGAACAACGAGAAUCAAGA |
| 170 | BMI1 | CAUGUAUGAGGAGGAACCU |
| 171 | BMI1 | CUAAUGGAUAUUGCCUACA |
| 172 | BMI1 | GGUUGAUACCUGAGACUGU |
| 173 | BMI1 | GACAUAACAGGAAACAGUA |
| 174 | BMI1 | GAGCCUUGCUUACCAGCAA |
| 175 | BMI1 | CCUUCUCUGCUAUGUCUGA |
| 176 | BMI1 | GGUCGAACUUGGUGUGUGU |
| 177 | BMI1 | CGAACUUGGUGUGUGUUCA |
| 178 | BMI1 | GUCUGCAAAAGAAGCACAA |
| 179 | BMI1 | CAGUACUAUGAAUGGAACC |
| 180 | BMI1 | CAGAUGGCAUUAUGCUUGU |
| 181 | BMI1 | GCUCGCAUUCAUUUUCUGC |
| 182 | BMI1 | CCCGCAGAAUAAAACCGAU |
| 183 | BMI1 | AGAUGGACUACAUGUGAUA |
| 184 | BMI1 | UCUGCAAAAGAAGCACAAU |
| 185 | BMI1 | CUGUAAAACGUGUAUUGUU |
| 186 | BMI1 | GGUAUAUGACAUAACAGGA |
| 187 | BMI1 | GGAAUAUGCCUUCUCUGCU |
| 188 | BMI1 | CUGCCAAUGGCUCUAAUGA |
| 189 | BMI1 | CAGCAGGUUGCUAAAAGAA |
| 190 | BMI1 | GAUGGACUACAUGUGAUAC |
| 191 | BMI1 | UAGUAUGAGAGGCAGAGAU |
| 192 | BMI1 | UUCAUUGAUGCCACAACCA |
| 193 | BMI1 | ACCAGCAAGUAUUGUCCUA |
| 194 | BMI1 | AGAACUGGAAAGUGACUCU |
| 195 | BMI1 | ACUAUCGUCCAAUUUGCUU |
| 196 | BMI1 | UCUGUUCCAUUAGAAGCAA |
| 197 | BMI1 | GUAAAAUGGACAUACCUAA |
| 198 | BMI1 | GCUGCUCUUUCCGGGAUUU |
| 199 | BMI1 | GAACAGAUUGGAUCGGAAA |
| 200 | BMI1 | CAUGUGACUAUCGUCCAAU |
| 201 | siCONT | CUUACGCUGAGUACUUCGA |
| | (negative | |
| | control | |
| | siRNA) | |

### Example 2

### Preparation of Double-Stranded Oligo RNA Molecules (SAMiRNA LP)

The double-stranded oligo RNA molecules (SAMiRNA LP) prepared in the present invention had a structure of the following Structural Formula (5).

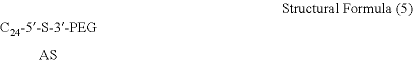

In Structural Formula (5), S is a sense strand of siRNA; AS is an antisense strand of the siRNA; PEG is a polyethylene glycol as a hydrophilic compound; C₂₄ is tetradocosane including a disulfide bond as a hydrophobic compound; and 5′ and 3′ mean orientations of ends of the double-stranded oligo RNA.

In the case of the sense strand of the siRNA in Structural Formula (5), oligo RNA-hydrophilic compound molecule comprising a sense strand of which polyethylene glycol was bound to a 3′-end region was synthesized by a method of connecting the phosphodiester bond configuring the backbone structure of the RNA using β-cyanoethyl phosphoramidite as described above while using polyethylene glycol (PEG, Mn=2,000)-CPG prepared according to a method in Example 1 disclosed in the existing Patent (KR 2012-0119212A) as the support, and then tetradocosane including the disulfide bond was bound to a 5′-end region, thereby preparing a sense strand of the desired RNA-polymer molecules. In the case of the antisense strand to be annealed with the strand, the antisense strand comprising the sequence complementary to that of the sense strand was prepared by the above-mentioned reaction.

After synthesizing was completed, the synthesized RNA single strand and the RNA polymer molecules were separated from the CPG by treating the reactants with ammonia (28% (v/v)) in a water bath at 60° C., and then a protective residue was removed by a deprotection reaction. The RNA single strand and the RNA polymer molecules from which the protective residue was removed were treated with N-methylpyrrolidone, triethylamine, and triethylaminetrihydrofluoride at a volume ratio of 10:3:4 in an oven at 70° C., thereby removing tert-butyldimethylsilyl (2′TBDMS).

The RNA was separated from the reactant and purified using a HPLC (LC918, Japan Analytical Industry, Japan) equipped with a Daisogel C18 (Daiso, Japan) column. Then, whether or not the purified RNA coincides with the desired base sequence was confirmed using a MALDI-TOF mass spectrometer (Shimadzu, Japan). Thereafter, in order to prepare each of the double-stranded oligo RNA polymer molecules, the same amount of sense and antisense strands were mixed and put into 1× annealing buffer (30 mM HEPES, 100 mM potassium acetate, 2 mM magnesium acetate, pH 7.0-7.5), followed by reacting with each other in a water bath at 90° C. for 3 minutes and reacting with each other again at 37° C., thereby preparing the double-stranded oligo RNA molecules containing siRNAs of the SEQ ID NOs. 1, 102, and 201, respectively (hereinafter, referred to as SAMiRNALP-Gank, SAMiRNALP-BMI, SAMiRNALP-CONT, respectively). It was confirmed through electrophoresis that the prepared double-stranded oligo RNA molecules were annealed.

### Example 3

### Preparation of Nanoparticles (SAMiRNA) Made of SAMiRNA LP and Measurement of Size

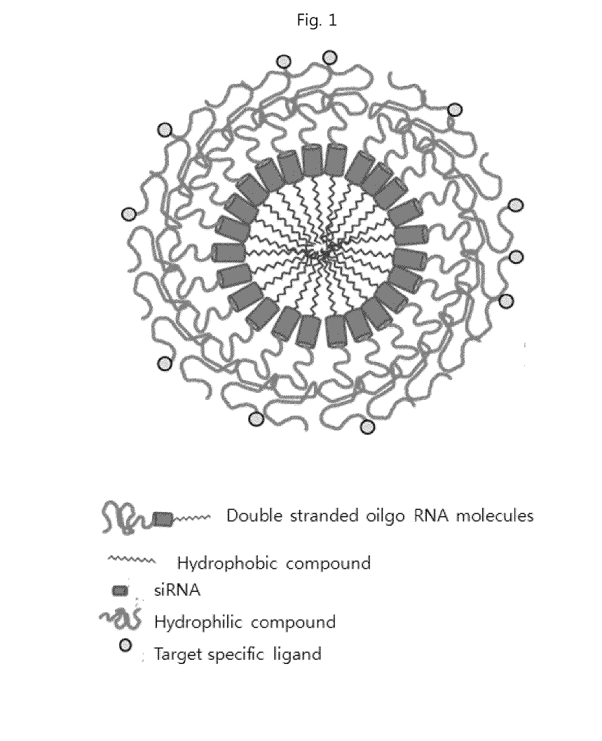

The SAMiRNA LP prepared in Example 2 formed nanoparticles, that is, micelles by hydrophobic interactions between the hydrophobic compounds bound to the ends of the double-stranded oligo RNA (See FIG. 1).

Sizes and polydispersity indexes (PDI) of nanoparticles made of SAMiRNALP-Gank, SAMiRNALP-BMI, and SAMiRNALP-CONT, respectively were analyzed, thereby confirming formation of the nanoparticles (SAMiRNA) made of the corresponding SAMiRNALP.

### Example 3-1

### Preparation of Nanoparticles

After dissolving SAMiRNALP-Gank in 1.5 ml Dulbecco's Phosphate Buffered Saline (DPBS) at a concentration of 50 μg/ml, nanoparticle powder was prepared by lyophilization at −75° C. and 5 mTorr for 48 hours and dissolved in the DPBS as a solvent, thereby preparing homogeneous nanoparticles. In the case of SAMiRNA-Gank+BMI, after dissolving each of the SAMiRNALP-Gank and SAMiRNA-BMI in 0.75 ml Dulbecco's Phosphate Buffered Saline (DPBS) at a concentration of 5 μg/ml, nanoparticle powders were prepared by lyophilization at −75° C. and 5 mTorr for 48 hours and dissolved in the DPBS as the solvent to prepare homogeneous nanoparticles, respectively, followed by mixing two compounds, thereby preparing nanoparticles containing the siRNAs comprising sense strand of the SEQ ID NOs. 1 and 102.

### Example 3-2

### Measurement of Sizes and Polydispersity Indexes (Hereinafter, Referred to as ‘PDI’) of Nanoparticles

The sizes of the nanoparticles were measured using a zeta-potential measurement. The sizes of the homogeneous nanoparticles prepared in Example 3-1 were measured using the zeta-potential measurement (Nano-ZS, MALVERN, UK). Here, a refractive index and absorption index for compounds were set to 1.459 and 0.001, respectively. In addition, a temperature of DPBS as the solvent was input as 25° C., and viscosity and a refractive index thereof were input as 1.0200 and 1.335, respectively. A one-time measurement consists of 15 repetitive size measurements, and this measurement was repeated six times. Sizes of the nanoparticles made of SAMiRNALP-BMI and SAMiRNALP-Gank+BMI were measured by the same method.

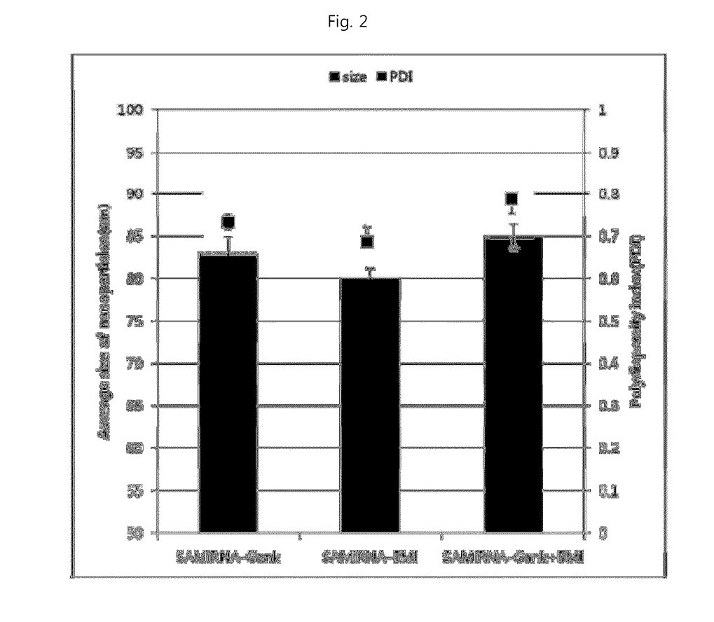

It was confirmed that the nanoparticles (SAMiRNA-Gank) made of the SAMiRNALP-Gank had a size of about 83 nm and a PDI value of 0.24, and the nanoparticles (SAMiRNA-BMI) made of the SAMiRNALP-BMI had a size of 80 nm and a PDI value of 0.22. It was confirmed that the nanoparticles (SAMiRNA-Gank+BMI) made of the SAMiRNALP-Gank+BMI had a size of about 85 nm and a PDI value of 0.26 (See FIG. 2). The PDI value is a value indicating that as the PDI value is decreased, the corresponding particles are more uniformly distributed. Therefore, it may be appreciated that the nanoparticles according to the present invention were formed to have a significantly uniform size.

### Example 4

### Confirmation of Target Gene Expression Inhibition in Human Liver Cancer Cell Lines (Hep3B Cell Lines) Using siRNA

The human liver cancer cell lines (Hep3B cell lines) were transfected using the siRNAs comprising sense strand of the SEQ ID NOs. 1 to 201 prepared in Example 1, respectively, and expression levels of the target genes in the transfected Hep3B cell lines were analyzed.

### Example 4-1

### Culture of Human Liver Cancer Cell Lines

The human liver cancer cell lines (Hep3B cell lines) obtained from American Type Culture Collection (ATCC) were cultured in an Eagle's minimum essential medium (EMEM, GIBCO/Invitrogen, USA) supplemented with 10% (v/v) fetal bovine serum, 100 units/ml penicillin, and 100 μg/ml streptomycin at 37° C. under 5% (v/v) CO₂ atmosphere.

### Example 4-2

### Transfection of the Desired siRNA in Human Liver Cancer Cell Lines

After 1×10⁵ Hep3B cell lines cultured in the Example 4-1 were cultured in a 12-well plate using the EMEM at 37° C. under 5% (v/v) CO₂ atmosphere for 18 hours, the medium was removed, and then 500 μl of Opti-MEM medium (GIBCO, US) was dispensed in each well.

Meanwhile, 1.5 μl of Lipofectamine™ RNAi Max (Invitrogen, US) and 248.5 μl of the Opti-MEM medium were mixed with each other to prepare a mixed solution and then reacted with each other at room temperature for 5 minutes. Then, 0.2 or 1 μl of each of the siRNAs (1 pmole/μl) of the SEQ ID NOs. 1 to 201 prepared in Example 1 was added to 230 μl of the Opti-MEM medium, thereby preparing a siRNA solution comprising a final concentration of 0.2 nM or 1 nM. The Lipofectamine™ RNAi Max mixed solution and the siRNA solution were mixed and then reacted with each other at room temperature for 20 minutes, thereby preparing a solution for transfection.

Thereafter, 500 μl of the solution for transfection was dispensed in each well containing tumor cell lines and the dispensed Opti-MEM medium and cultured for 6 hours, followed by removal of the Opti-MEM medium. Here, 1 ml of the EMEM medium was dispensed in each well and cultured at 37° C. under 5% (v/v) CO₂ atmosphere for 24 hours.

### Example 4-3

### Quantitative Analysis of Target Gene mRNA

Total RNA was extracted from the cell lines transfected in the example 4-2 to prepare cDNA, and then a target gene mRNA expression level was relatively quantified using a real-time polymerase chain reaction (PCR).

### Example 4-3-1

### Separation of RNA from Transfected Cells and Preparation of cDNA

Total RNA was extracted from the cell lines transfected in the example 4-2 by using an RNA extraction kit (AccuPrep Cell total RNA extraction kit, Bioneer, Korea), and cDNA was prepared from the extracted RNA using an RNA reverse transcriptase (AccuPower CycleScript RT Premix/dT20, Bioneer, Korea), as follows. More specifically, 1 μg of the extracted RNA was put into each of the 0.25 ml Eppendorf tubes containing AccuPower CycleScript RT Premix/dT20 (Bioneer, Korea), and distilled water treated with diethyl pyrocarbonate (DEPC) was added so as to have a total volume of 20 μl. Two steps of RNA-primer hybridization at 30° C. for 1 minute and preparation of cDNA at 52° C. for 4 minutes were repeated six times using a PCR machine (MyGenie™ 96 Gradient Thermal Block, Bioneer, Korea), and then the amplification reaction was terminated by inactivating enzymes at 95° C. for 5 minutes.

### Example 4-3-2

### Relative Quantitative Analysis of Target Gene mRNA

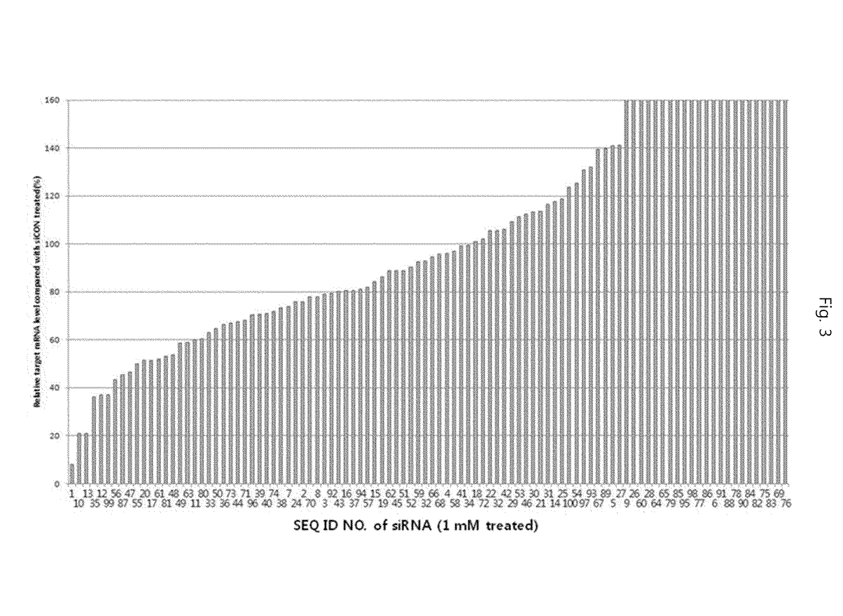

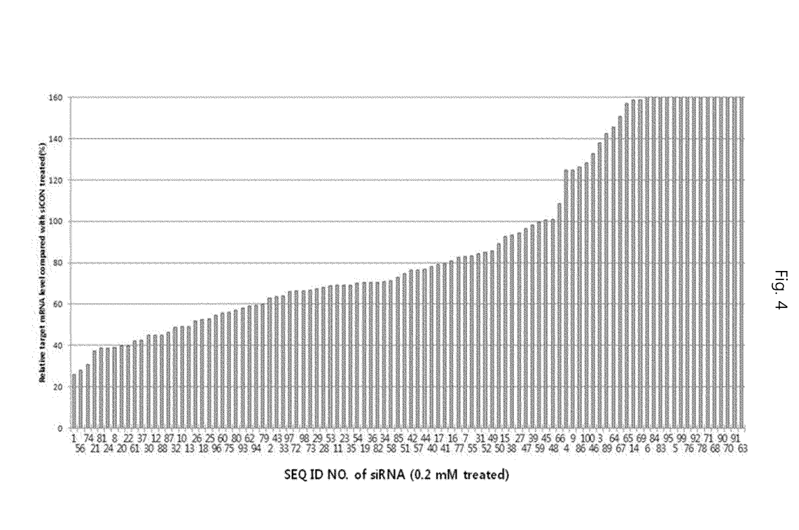

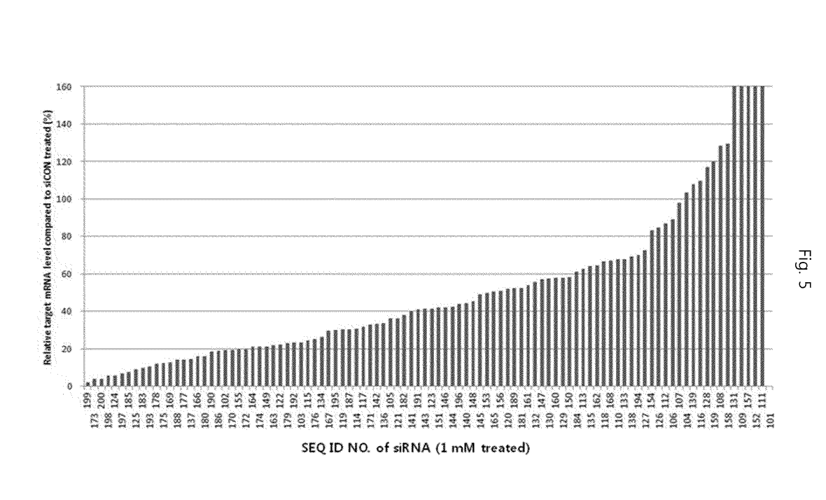

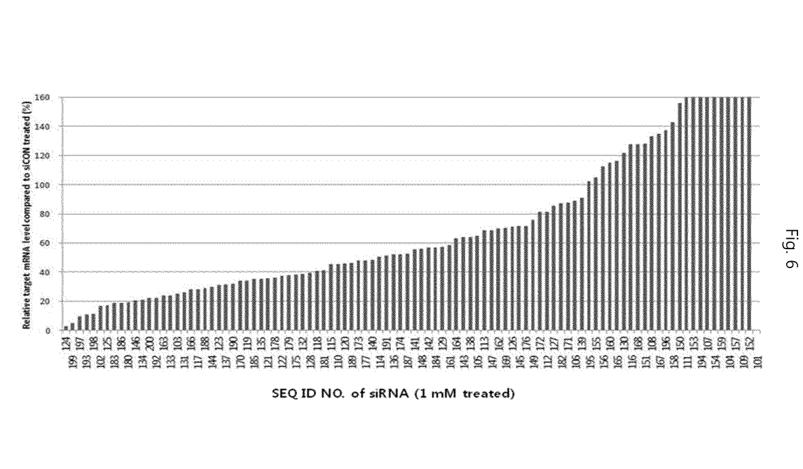

The relative level of liver cancer related gene mRNA was quantified through the real-time PCR using the cDNA prepared in the example 4-3-1 as a template as follows. The cDNA prepared in the example 4-3-1 was diluted 5 times with distilled water in each well of a 96-well plate, and then in order to accurately analyze the target gene mRNA expression level, 3 μl of the diluted cDNA, 10 μl of 2× GreenStar™ PCR master mix (Bioneer, Korea), 6 μl of distilled water, and 1 μl of Gankyrin qPCR primers (each of F and R: 10 pmole/μl, Bioneer, Korea, See Table 2) were used to prepare a mixed solution. Meanwhile, in order to normalize the target gene mRNA expression level, glyceraldehyde 3-phosphate dehydrogenase (GAPDH), which is a housekeeping gene (hereinafter, referred to as HK gene), was used as a reference gene. The following reaction was performed on the 96-well plate containing the mixed solution using an Exicycler™96 Real-Time Quantitative Thermal Block (Bioneer, Korea). Enzyme activation and a secondary structure of cDNA were removed by performing the reaction at 95° C. for 15 minutes. Then, four steps of denaturing at 94° C. for 30 seconds, annealing at 58° C. for 30 seconds, extension at 72° C. for 30 seconds, and SYBR green scan were repetitively performed 42 times, and then a final extension was performed at 72° C. for 3 minutes. Thereafter, the temperature was maintained at 55° C. for 1 minute, and a melting curve of 55° C.˜95° C. was analyzed. After finishing the PCR, each of the obtained threshold cycle (Ct) values of the target genes was corrected using the GAPDH gene, thereby obtaining the corrected Ct value of the target gene. Then, a difference (ΔCt) in Ct value was calculated using an experimental group treated with the siRNA (siCONT) comprising a control sequence that does not inhibit gene expression as a control group. The expression levels of the target genes in the cells treated with Gankyrin specific siRNAs comprising sense strand of SEQ ID NOs. 1 to 100 were relatively quantified, respectively, using the ΔCt values and the calculation equation of 2(−ΔCt)×100 (See FIGS. 3 and 4). In addition, in each of the experimental groups treated with BMI-1 specific siRNAs (SEQ ID NO. 101 to 200 as a sense strand), mRNA of the target gene was relatively quantified by the same method using the BMI-1 qPCR primer and the GAPDH qPCR primer (FIGS. 5 and 6). In order to select the siRNA comprising high efficiency, the siRNAs used in the case in which the mRNA expression levels for each gene at the concentrations of 0.2 nM and 1 nM were commonly significantly decreased were selected (SEQ ID NOs. 1, 10, 13, 56, 99, 102, 180, 197, 199, and 200 as a sense strand).

TABLE 2
qPCR primer sequence information
(F: forward primer, R: reverse primer)

| | | SEQ |
|---|---|---|
| Name | Sequence | ID NO. |
| GAPDH-F | GGTGAAGGTCGGAGTCAACG | 202 |
| GAPDH-R | ACCATGTAGTTGAGGTCAATGAAGG | 203 |
| Gankyrin-F | AGCAGCCAAGGGTAACTTGA | 204 |
| Gankyrin-R | CACTTGCAGGGGTGTCTTTT | 205 |
| BMI1-F | TCATCCTTCTGCTGATGCTG | 206 |
| BMI1-R | CCGATCCAATCTGTTCTGGT | 207 |

### Example 5

### Selection of siRNA Comprising High Efficiency in Human Liver Cancer Cell Lines (Hep3B and Huh-7 Cell Lines) and Measurement of Inhibition Concentration 50% (IC50)

The human liver cancer cell lines (Hep3B and Huh-7) were transfected using the siRNAs comprising sense strand of the SEQ ID NOs. 1, 10, 13, 56, 99, 102, 180, 197, 199, 200, and 201 selected in Examples 4-3-2, and expression levels of the target gene in the transfected human liver cancer cell lines (Hep3B and Huh-7 cell lines) were analyzed, thereby selecting the siRNA comprising the high efficiency. Then, performance of the siRNA was confirmed by measuring IC50 of the siRNA comprising the highest efficiency.

### Example 5-1

### Culture of Human Liver Cancer Cell Lines

The human liver cancer cell lines (Hep3B cell lines) obtained from American Type Culture Collection (ATCC) were cultured under the same condition as that in Example 4-1.

The human liver cancer cell lines (Huh-7 cell lines) obtained from Korean Cell Line Bank (KCLB) were cultured in an RPMI-1640 culture medium (GIBCO/Invitrogen, USA) supplemented with 10% (v/v) fetal bovine serum, 100 units/ml penicillin, and 100 μg/ml streptomycin at 37° C. under 5% (v/v) CO₂ atmosphere.

### Example 5-2

### Transfection of the Desired siRNA in Human Liver Cancer Cell Lines

After the Hep3B cell lines cultured in Example 5-1 were cultured under the same condition as that in Example 4-2, 1.5 μl of Lipofectamine™ RNAi Max (Invitrogen, US) and 248.5 μl of the Opti-MEM medium were mixed with each other to prepare a mixed solution and then reacted with each other at room temperature for 5 minutes. Then, 0.04, 0.2 or 1 μl of each of the siRNAs (1 pmole/μl) comprising sense strand of the SEQ ID NOs. 1, 10, 13, 56, 99, and 201 prepared in Example 1 and the siRNA (Gank_Ref, GGGCAGCAGCCAAGGGUAA (SEQ ID No. 208), Dharmacon A1, 1 pmole/μl) according to the related art (US 2008/0071075) was added to 230 μl of the Opti-MEM medium, thereby preparing a siRNA solution comprising a final concentration of 0.04, 0.2 or 1 nM. 0.008, 0.04, or 0.2 μl of each of the siRNAs (1 pmole/μl) comprising sense strand of the SEQ ID NOs. 102, 180, 197, 199, 200, and 201 prepared in Example 1 and the siRNA (1 pmole/μl) according to the related art was added to 230 μl of the Opti-MEM medium, thereby preparing a siRNA solution comprising a final concentration of 0.008, 0.04, or 0.2 nM. The Lipofectamine™ RNAi Max mixed solution and the siRNA solution were mixed and reacted with each other at room temperature for 20 minutes, thereby preparing a solution for transfection.

In addition, after 1×10⁵ Huh-7 cell lines cultured in the Example 5-1 were cultured in a 12-well plate using the RPMI-1640 culture medium at 37° C. under 5% (v/v) CO₂ atmosphere for 18 hours, the medium was removed, and then 500 μl of the Opti-MEM medium (GIBCO, US) was dispensed in each well. Meanwhile, 1.5 μl of Lipofectamine™ RNAi Max (Invitrogen, US) and 248.5 μl of the Opti-MEM medium were mixed with each other to prepare a mixed solution and then reacted with each other at room temperature for 5 minutes.

Then, 0.04, 0.2 or 1 μl of each of the siRNAs (1 pmole/μl) comprising sense strand of the SEQ ID NOs. 1, 10, 13, 56, 99, and 201 prepared in Example 1 and the siRNA (Gank_Ref, 1 pmole/μl) according to the related art was added to 230 μl of the Opti-MEM medium, thereby preparing a siRNA solution comprising a final concentration of 0.04, 0.2 or 1 nM.

0.008, 0.04, or 0.2 μl of each of the siRNAs (1 pmole/μl) comprising sense strand of the SEQ ID NOs. 102, 180, 197, 199, 200, and 201 prepared in Example 1 and the siRNA (BMI-1_Ref, CGTGTATTGTTCGTTACCT, (SEQ ID No. 209), Cancer Sci. 2010 February; 101(2):379-86) (1 pmole/μl) was added to 230 μl of the Opti-MEM medium, thereby preparing a siRNA solution comprising a final concentration of 0.008, 0.04, or 0.2 nM. The Lipofectamine™ RNAi Max mixed solution and the siRNA solution were mixed and reacted with each other at room temperature for 20 minutes, thereby preparing a solution for transfection.

Thereafter, 500 μl of the solution for transfection was dispensed in each well containing tumor cell lines and the dispensed Opti-MEM medium and cultured for 6 hours, followed by removal of the Opti-MEM medium. Here, 1 ml of the RPMI 1640 medium was dispensed in each well and cultured at 37° C. under 5% (v/v) CO₂ atmosphere for 24 hours.

### Example 5-3

### Quantitative Analysis of Target Gene mRNA

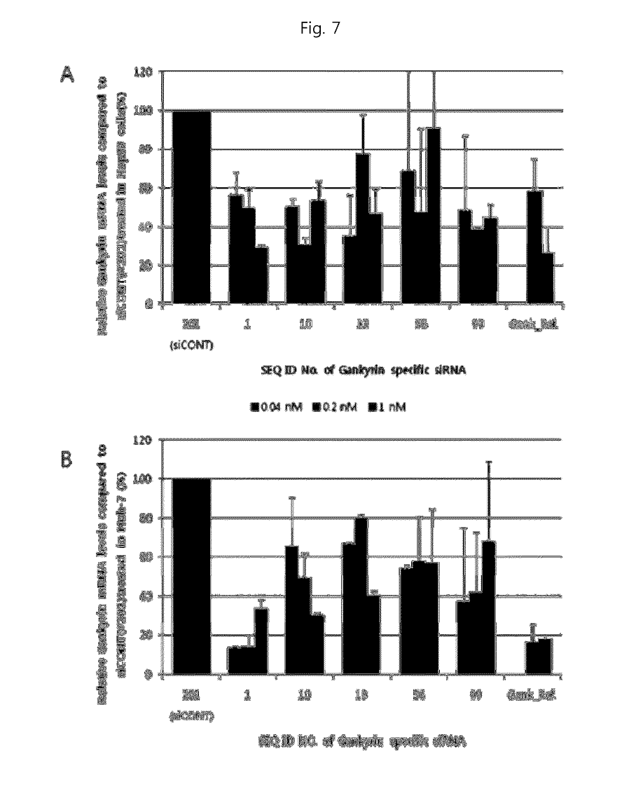

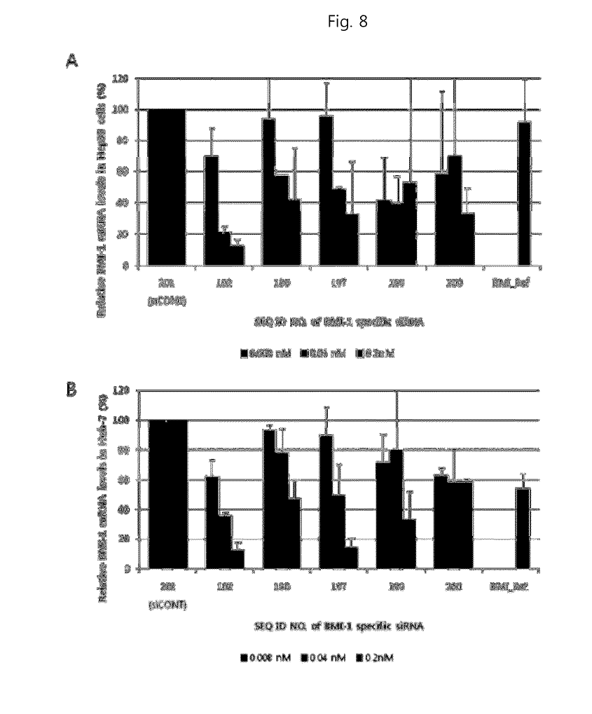

Total RNA was extracted from the cell lines transfected in the example 5-2 to prepare cDNA, and then a target gene mRNA expression level was relatively quantified using a real-time PCR by the same method as that in Example 4-3 (FIGS. 7A to 8B). Effectiveness of each of the siRNAs may be clearly confirmed by observing the target gene expression inhibition level in two kinds of liver cancer cells. It was confirmed that the siRNAs comprising sense strand of the SEQ ID NOs. 1, 10, 13, 102, 197, and 199 had relatively high target gene expression inhibition levels even at a significantly low concentration.

### Example 5-4

### Measurement of IC50

One kind of siRNAs was selected from the high efficiency siRNAs confirmed in Example 5-3 with respect to each of the genes, and performance of the corresponding siRNA was confirmed by confirming an IC50. After the Hep3B cell lines cultured in Example 5-1 were cultured under the same condition as that in Example 4-2, 1.5 μl of Lipofectamine™ RNAi Max (Invitrogen, US) and 248.5 μl of the Opti-MEM medium were mixed with each other to prepare a mixed solution and reacted with each other at room temperature for 5 minutes. Then, 0.8 or 0.4 μl of each of the siRNAs (0.01 pmole/μl) of the SEQ ID NOs. 1, 102, and 201 prepared in Example 1 or 0.2, 1, or 5 μl of each of the siRNAs (1 pmole/μl) comprising sense strand of the SEQ ID NOs. 1, 102, and 201 was added to 230 μl of the Opti-MEM medium, thereby preparing a siRNA solution comprising a final concentration of 8 pM, 40 pM, 0.2 nM, 1 nM, or 5 nM. The Lipofectamine™ RNAi Max mixed solution and the siRNA solution were mixed and reacted with each other at room temperature for 20 minutes, thereby preparing a solution for transfection.

In addition, after 1×10⁵ Huh-7 cell lines cultured in the Example 5-1 were cultured in a 12-well plate using the RPMI-1640 culture medium at 37° C. under 5% (v/v) CO₂ atmosphere for 18 hours, the medium was removed, and then 500 μl of the Opti-MEM medium (GIBCO, US) was dispensed in each well. Meanwhile, 1.5 μl of Lipofectamine™ RNAi Max (Invitrogen, US) and 248.5 μl of the Opti-MEM medium were mixed with each other to prepare a mixed solution and then reacted with each other at room temperature for 5 minutes. Then, 0.8 or 0.4 μl of each of the siRNAs (0.01 pmole/μl) comprising sense strand of the SEQ ID NOs. 1, 102, and 201 prepared in Example 1 or 0.2, 1, or 5 μl of each of the siRNAs (1 pmole/μl) comprising sense strand of the SEQ ID NOs. 1, 102, and 201 was added to 230 μl of the Opti-MEM medium, thereby preparing a siRNA solution comprising a final concentration of 8 pM, 40 pM, 0.2 nM, 1 nM, or 5 nM. The Lipofectamine™ RNAi Max mixed solution and the siRNA solution were mixed and then reacted with each other at room temperature for 20 minutes, thereby preparing a solution for transfection.

Thereafter, 500 μl of the solution for transfection was dispensed in each well containing tumor cell lines and the dispensed Opti-MEM medium and cultured for 6 hours, followed by removal of the Opti-MEM medium. Here, 1 ml of the RPMI 1640 culture medium was dispensed in each well and cultured at 37° C. under 5% (v/v) CO₂ atmosphere for 24 hours.

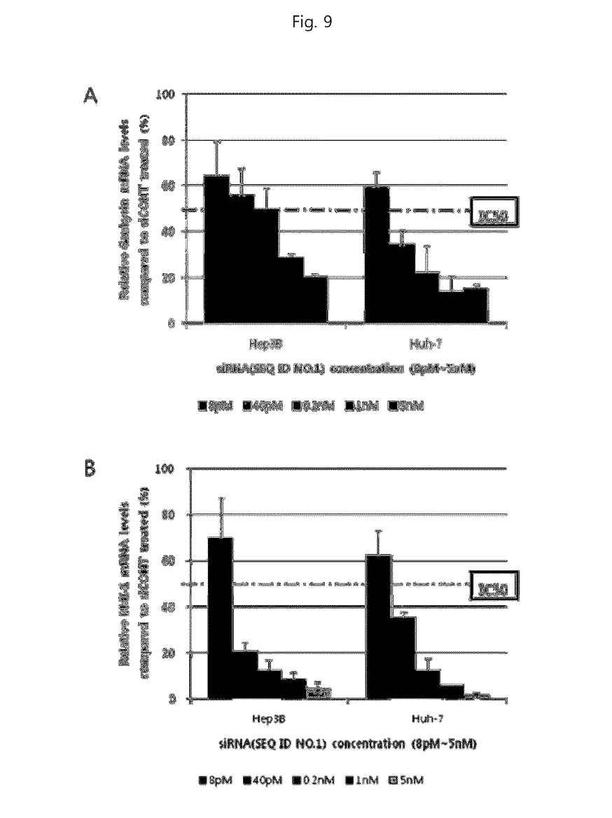

Total RNA was extracted from the transfected cell lines to prepare cDNA, and then a target gene mRNA expression level was relatively quantified using a real-time PCR by the same method as that in Example 4-3 (FIGS. 9A and 9B). It was observed that the IC50 of the siRNA comprising sense strand of SEQ ID NO. 1 was 40 to 200 pM in the Hep3B cell lines and 8 to 40 pM in the Huh-7 cell lines, and the IC50 of the siRNA comprising sense strand of SEQ ID NO. 102 was 8 to 40 pM in both of the Hep3B and Huh-7 cell lines. Therefore, it was confirmed that the siRNA selected in the present invention had high efficiency.

### Example 6

### Colony Forming Assay for Confirming Inhibition Effect of Gankyrin or BMI-1 Specific siRNA

A method of measuring transformation of cells by performing a colony forming assay on a single cell in vitro is a semi-quantitative method and is derived from lost of contact inhibition by the cancer cell and anchorage independent phenotypic characterizations of the cancer cell. This assay method is used to confirm survival of cancer cells by a specific anticancer drug in vitro in the case in which the cancer cells were treated with the corresponding anticancer drug (Clonogenic Assay of Cells in Vitro, Nat. Protoc. 1(5): 2315-9, 2006).

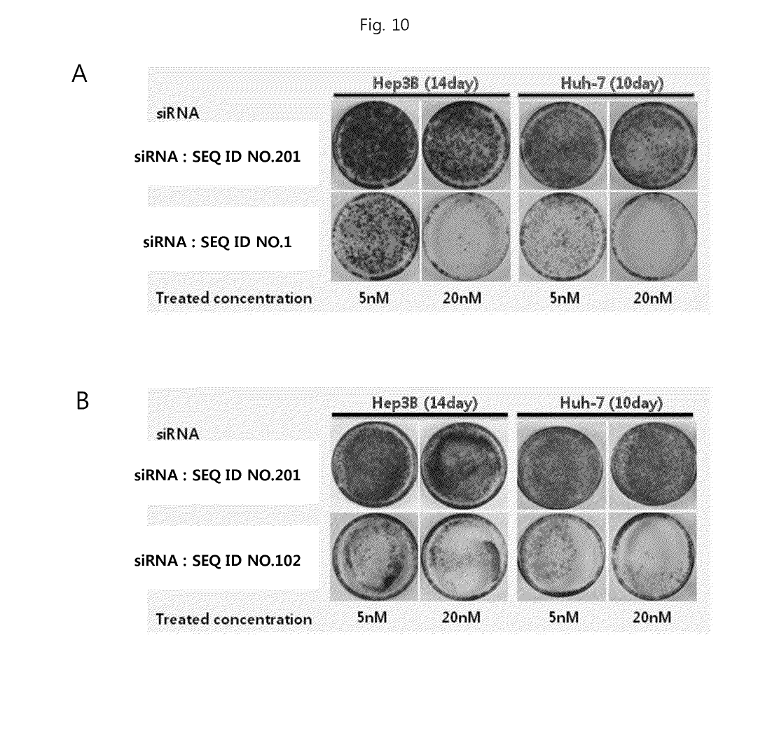

In order to confirm how much colony forming of the cancer cells was inhibited by the high efficiency Gankyrin or BMI-1 specific siRNA selected in Example 5-4, the colony forming assay (CFA) was performed. The hep3B and Huh-7 cell lines cultured in Example 5-1 were inoculated in a 35 mm Petri-dish (1×104/dish), respectively. After 20 hours, the cells were transfected at a concentration of 5 nM or 20 nM by the same method as that in Example 5-4. The culture medium of the transfected cells was replaced once every three days, and after 10 to 14 days of the transfection, the cells were stained with Diff Quik (Sysmex, Japan) to compare colony forming degrees with each other (FIGS. 10A and 10B). It may be confirmed that in groups treated with the siRNAs of the SEQ ID NO. 1 and 102, colonies were concentration-dependently formed at a significantly low level as compared to the control group treated with the siRNA comprising sense strand of SEQ ID NO. 201.

### Example 7

### Confirmation of Target Gene Expression Inhibition and Cell Growth Inhibition by Combination of Gankyrin Specific siRNA and BMI-1 Specific siRNA

Cells were transfected with a combination of the high efficiency siRNAs of the SEQ ID NOs. 1 and 102 confirmed in Example 5-4 at a concentration of 5 or 20 nM, which was a concentration higher than the IC50. Then, in the case in which expression of two genes were simultaneously inhibited, target gene expression inhibition levels and a synergic effect on cell growth inhibition were confirmed.

### Example 7-1

### Transfection of the Desired siRNA in Human Liver Cancer Cell Lines

After 1×10⁵ Huh-7 cell lines cultured in the Example 5-1 were cultured in a 12-well plate using the RPMI-1640 culture medium at 37° C. under 5% (v/v) CO₂ atmosphere for 18 hours, the medium was removed, and then 500 μl of the Opti-MEM medium (GIBCO. US) was dispensed in each well. Meanwhile, 1.5 μl of Lipofectamine™ RNAi Max (Invitrogen, US) and 248.5 μl of the Opti-MEM medium were mixed with each other to prepare a mixed solution and reacted with each other at room temperature for 5 minutes. Then, 5 μl of each of the siRNAs (1 pmole/μl) comprising sense strand of the SEQ ID NOs. 1, 102, and 201 prepared in Example 1 was added to 230 μl of the Opti-MEM medium, thereby preparing a siRNA solution comprising a final concentration of 5 nM. The Lipofectamine™ RNAi Max mixed solution and the siRNA solution were mixed and reacted with each other at room temperature for 20 minutes, thereby preparing a solution for transfection.

Thereafter, 500 μl of the solution for transfection was dispensed in each well containing tumor cell lines and the dispensed Opti-MEM medium and cultured for 6 hours, followed by removal of the Opti-MEM medium. Here, 1 ml of the RPMI 1640 culture medium was dispensed in each well and cultured at 37° C. under 5% (v/v) CO₂ atmosphere for 24 hours.

### Example 7-2

### Quantitative Analysis of Target Gene mRNA by Combination of Gankyrin Specific siRNA and BMI-1 Specific siRNA

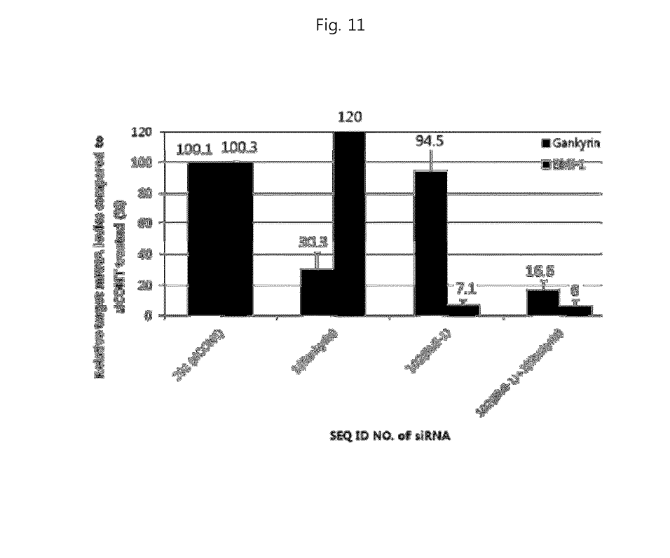

Total RNA was extracted from the cell lines transfected in the example 7-1 to prepare cDNA, and then a target gene mRNA expression level was relatively quantified using a real-time PCR by the same method as that in Example 4-3 (FIG. 11). It may be confirmed by observing the target gene expression inhibition level in the human liver cancer cell lines (Huh-7 cell lines) that expression of the target genes was inhibited by the siRNAs simultaneously used. Particularly, it may be observed that even in the case in which the siRNAs comprising sense strand of the SEQ ID NOs. and 102 were simultaneously used, expression of the target genes were simultaneously inhibited.

### Example 7-3

### Confirmation of Cell Growth Inhibition by Combination of Gankyrin Specific siRNA and BMI-1 Specific siRNA

Cell growth inhibition was confirmed through the target gene expression inhibition by the combination of the Gankyrin specific siRNA and the BMI-1 specific siRNA.

After the Hep3B cell lines cultured in Example 4-1 were cultured under the same condition as in Example 4-2, the medium was removed, and 500 μl of the Opti-MEM medium (GIBCO, US) was dispensed in each well. Meanwhile, 1.5 μl of Lipofectamine™ RNAi Max (Invitrogen, US) and 248.5 μl of the Opti-MEM medium were mixed with each other to prepare a mixed solution and reacted with each other at room temperature for 5 minutes. Then, 5 or 20 μl of each of the siRNAs (1 pmole/μl) of the SEQ ID NOs. 1, 102, and 201 prepared in Example 1 was added to 230 μl of the Opti-MEM medium, thereby preparing a siRNA solution comprising a final concentration of 5 or 20 nM. The Lipofectamine™ RNAi Max mixed solution and the siRNA solution were mixed and reacted with each other at room temperature for 20 minutes, thereby preparing a solution for transfection.

Thereafter, 500 μl of the solution for transfection was dispensed in each well containing tumor cell lines and the dispensed Opti-MEM medium and cultured for 6 hours, followed by removal of the Opti-MEM medium. Here, 1 ml of the RPMI 1640 culture medium was dispensed in each well and cultured at 37° C. under 5% (v/v) CO₂ atmosphere for 72 hours.

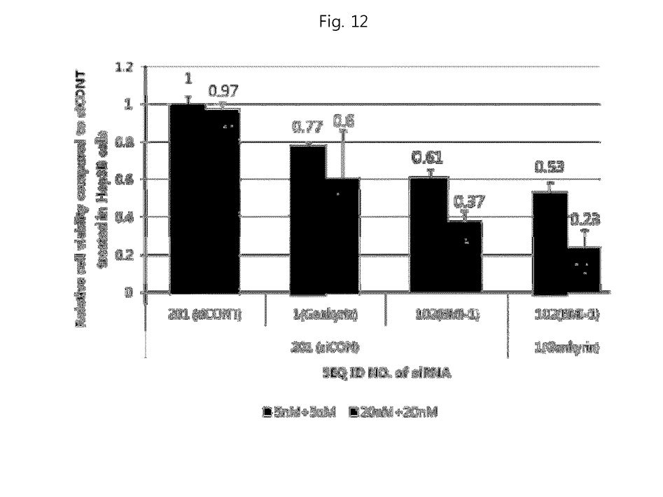

Cell viability was confirmed by comparing the number of cells with that in the Experimental group treated with the siRNA comprising sense strand of SEQ ID NO. 201 (FIG. 12). It may be confirmed that in the case in which the cell lines treated with the siRNAs of the SEQ ID NO. 1 and 102 at the same time, cell viability was concentration-dependently decreased, and the growth suppression effect was more excellent than a growth suppression effect exhibited when expression of any one gene was suppressed.

### Example 8

### Confirmation of Target Gene Expression Inhibition and Liver Cancer Growth Inhibition by Nanoparticles Containing Gankyrin and/or BMI-1 Specific siRNA in Animal Model

In order to confirm effects of the selected Gankyrin or BMI-1 specific siRNA in vivo, nanoparticles made of double-stranded oligo RNA molecules were prepared and then injected into a mouse liver cancer model. Then, target gene expression inhibition and liver cancer growth inhibition were confirmed.

### Example 8-1

### Preparation of Mouse Liver Cancer Model (Orthotropic Liver Cancer Model)

The human liver cancer cell lines (Hep3B cell lines, 2×10⁶) cultured in Example 4-1 were transplanted into the liver (left hepatic lobe) in Balb/c nude mice to establish liver cancer models. Next, it was confirmed that cancer cell were formed by measuring a α-Fetoprotein (AFP) level, which is a liver cancer marker in serum. When the AFP level in serum became about 1,000 ng/ml, five mice were allocated to each experimental group according to the level of AFP.

### Example 8-2

### Target Gene Expression Inhibition by Nanoparticles (SAMiRNA) Made of Double-Stranded Oligo RNA Molecules

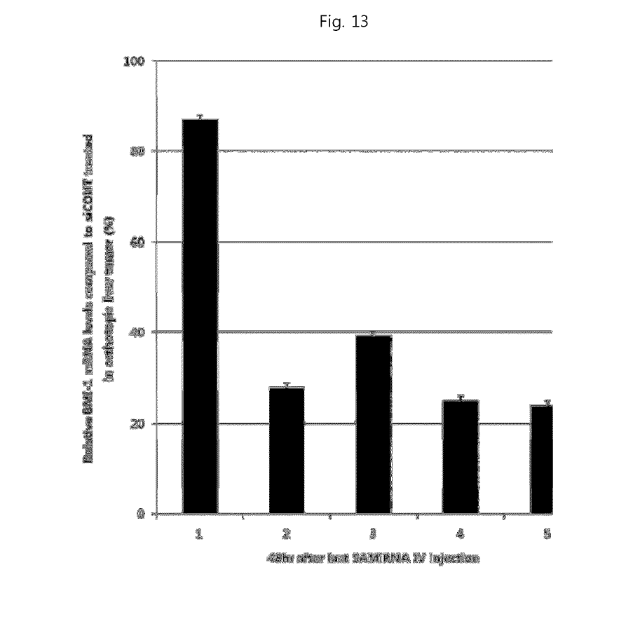

Homogeneous nanoparticles were prepared by the method in Example 3-1 using the double-stranded oligo RNA molecules (SAMiRNA LP) containing the siRNAs of the SEQ ID NOs. 102 and 201 synthesized in Example 2. Nanoparticles (SAMiRNA-CONT) containing the siRNA of SEQ ID NO. 201 were set as a control group, and nanoparticles (SAMiRNA-BMI) containing the siRNA comprising sense strand of SEQ ID NO. 102 were set as an experimental group. The nanoparticles were administered at 5 mg/kg body weight, and 100 μl of the prepared nanoparticles in DPBS was intravenously injected twice into the mouse liver cancer models prepared in Example 8-1 using a 1 ml syringe (0.25 mm×8 mm, 31 Gauge, BD328820, USA). In order to increase reliability, a blind test was performed. After 48 hours of the last injection, liver cancer tissue of the mice was separated. Total RNA was extracted from the separated cancer tissue to prepare cDNA, and then a target gene mRNA expression level was relatively quantified using a real-time PCR by the same method as that in Example 4-3 (FIG. 13). In an individual, expression inhibition was delayed, but it was confirmed that expression of BMI-1, which was the target gene, was inhibited at a level of average 70% except for the individual and the target gene expression was inhibited at a level of maximum 80% in some individuals (individual numbers 2, 4, and 5). Therefore, it was confirmed that the nanoparticles made of the double-stranded oligo RNA molecules according to the present invention had an excellent target gene expression inhibition effect in vivo.

### Example 8-3

### Confirmation of Liver Cancer Growth Inhibition by Nanoparticles (SAMiRNA) Made of Double-Stranded Oligo RNA Molecules

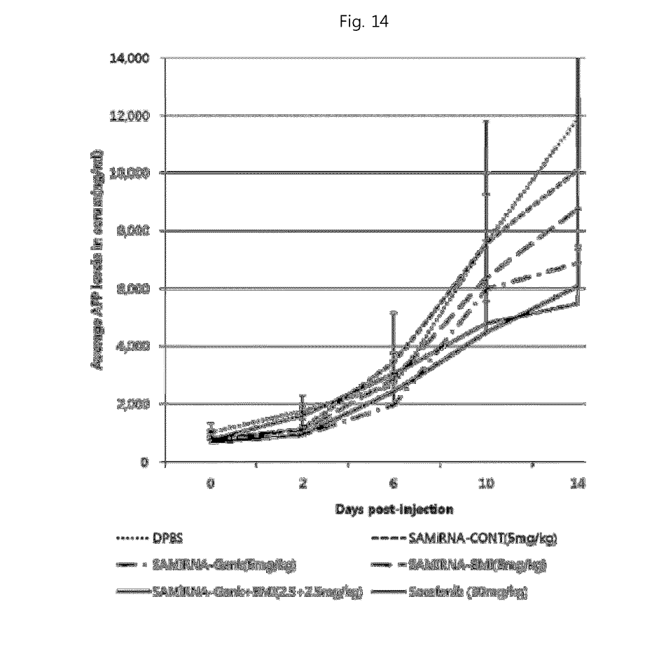

Homogeneous nanoparticles were prepared by the method in Example 3-1 using the double-stranded oligo RNA molecules (SAMiRNA LP) containing the siRNAs comprising sense strand of the SEQ ID NOs. 1, 102, and 201 synthesized in Example 2. Groups treated with DPBS, which was a solvent, and the nanoparticles (SAMiRNA-CONT) containing the siRNA of SEQ ID NO. 201, respectively, were set as negative control groups, groups treated with the nanoparticles (SAMiRNA-Gank) containing the siRNA comprising sense strand of SEQ ID NO. 1, the nanoparticles (SAMiRNA-BMI) containing the siRNA of SEQ ID NO. 102, and the nanoparticles (SAMiRNA-Gank+BMI) prepared by mixing the double-stranded oligo RNA molecules containing the siRNA comprising sense strand of SEQ ID NO. 1 and the double-stranded oligo RNA molecules containing the siRNA comprising sense strand of SEQ ID NO. 102 at the same content, respectively, were set as experimental groups, and a group treated with sorapenib, which is a kinase inhibitor, was set as a positive control group. The nanoparticles were prepared so as to be injected at 5 mg/kg body weight, and 100 μl of the prepared nanoparticles in DPBS was intravenously injected 14 times into the mouse liver cancer models prepared in Example 8-1 for 2 weeks using a 1 ml syringe (0.25 mm×8 mm, 31Gauge, BD328820, USA). In order to increase reliability, a blind test was performed. In the positive control group, sorapenib was orally administered at 30 mg/kg body weight 14 times to the mouse liver cancer model prepared in Example 8-1 for 2 weeks. After 2, 6, 10, and 14 days of initial injection, a growth level of cancer was confirmed by measuring the AFP level in blood (FIG. 14). In the experimental group into which the nanoparticles containing the Gankyrin or BMI-1 specific siRNA were injected, the AFP level in blood was decreased by about 20 to 30% as compared to the control group, and in the experimental group into which the nanoparticles simultaneously containing the Gankyrin and BMI-1 specific siRNAs were injected, the AFP level was slightly lower than that in the positive control group after 10 days of the initial injection and decreased by about 40% as compared to the negative control group after 14 days. Therefore, it may be confirmed that the nanoparticles made of the double-stranded oligo RNA molecules containing the Gankyrin and/or BMI-1 specific siRNA had an excellent anti-cancer effect.

1. A Gankyrin or BMI-1 specific siRNA comprising a sense strand comprising any one sequence selected from SEQ ID NOs. 1 to 200 and an antisense strand comprising a sequence complementary thereto.

2. The siRNA of claim 1, wherein the sense or antisense strand of the siRNA is composed of 19 to 31 nucleotides.

3. The siRNA of claim 1, composed of a sense strand comprising any one sequence selected from a group consisting of SEQ ID NOs. 1, 10, 13, 56, 99, 102, 180, 197, 199, and 200 and an antisense strand comprising a sequence complementary thereto.

4. The siRNA of claim 1, wherein the sense or antisense strand of the siRNA includes at least one chemical modification.

5. The siRNA of claim 4, wherein the chemical modification is at least one selected from modification by substitution of —OH group with —CH₃ (methyl), —OCH3 (methoxy), —NH2, —F (fluorine), —O-2-methoxyethyl, —O-propyl, —O-2-methylthioethyl, —O-3-aminopropyl, —O-3-dimethylaminopropyl, —O—N-methylacetamido, or —O-dimethylamidooxyethyl at a 2′-carbon site of a sugar structure in a nucleotide;
modification by substitution of oxygen in a sugar structure in the nucleotide with sulfur;
modification of a nucleotide bond into a phosphorothioate bond, a boranophosphate bond, or a methyl phosphonate bond; and
modification into a peptide nucleic acid (PNA) type, a locked nucleic acid (LNA) type, or a unlocked nucleic acid (UNA) type.

6. The siRNA of claim 1, wherein at least one phosphate group(s) is bound to a 5′-end of the antisense strand of the siRNA.

7. Double-stranded oligo RNA molecule(s) comprising a structure of the following Structural Formula (1).
A-X—R—Y—B Structural Formula (1)
where A is a hydrophilic compound, B is a hydrophobic compound, X and Y each are independently a simple covalent bond or a linker-mediated covalent bond, and R is Gankyrin or BMI-1 specific siRNA.

8. The double-stranded oligo RNA molecule(s) of claim 7, having a structure of Structural Formula (2) 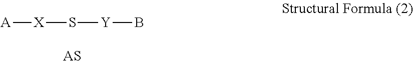
where S is the sense strand of the siRNA of claim 7, AS is the antisense strand thereof, and A, B, X, and Y have the same definitions as in claim 7.

9. The double-stranded oligo RNA molecule(s) of claim 8, having a structure of Structural Formula (3) 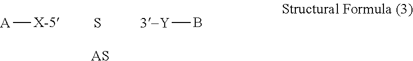
where A, B, X, Y, S, and AS have the same definitions as those in claim 8, and 5′ and 3′ mean a 5′-end and a 3′-end of the sense strand of the siRNA, respectively.

10. The double-stranded oligo RNA molecule(s) of claim 7, wherein the Gankyrin or BMI-1 specific siRNA comprises a sense strand comprising any one sequence selected from SEQ ID NOs. 1 to 200 and an antisense strand comprising a sequence complementary thereto.

11. The double-stranded oligo RNA molecule(s) of claim 7, wherein the hydrophilic compound has a molecular weight of 200 to 10,000.

12. The double-stranded oligo RNA molecule(s) of claim 11, wherein the hydrophilic compound is any one selected from a group consisting of polyethylene glycol (PEG), polyvinyl pyrrolidone, and polyoxazoline.

13. The double-stranded oligo RNA molecule(s) of claim 7, wherein the hydrophobic compound has a molecular weight of 250 to 1,000.

14. The double-stranded oligo RNA molecule(s) of claim 13, wherein the hydrophobic compound is any one selected from a group consisting of a steroid derivative, a glyceride derivative, glycerol ether, polypropylene glycol, saturated or unsaturated C12-C50 hydrocarbon, diacylphosphatidylcholine, fatty acid, phospholipid, and lipopolyamine.

15. The double-stranded oligo RNA molecule(s) of claim 14, wherein the steroid derivative is selected from a group consisting of cholesterol, cholestanol, cholic acid, cholesteryl formate, cholestanyl formate, and cholestearyl amine.

16. The double-stranded oligo RNA molecule(s) of claim 14, wherein the glyceride derivative is selected from mono-, di-, and tri-glycerides.

17. The double-stranded oligo RNA molecule(s) of claim 7, wherein the covalent bond represented by X and Y is a non-degradable bond or a degradable bond.

18. The double-stranded oligo RNA molecule(s) of claim 17, wherein the non-degradable bond is an amide bond or a phosphate bond.

19. The double-stranded oligo RNA molecule(s) of claim 17, wherein the degradable bond is a disulfide bond, an acid-degradable bond, an ester bond, an anhydride bond, a biodegradable bond, or an enzyme-degradable bond.

20. The double-stranded oligo RNA molecule(s) of claim 7, wherein a ligand which binds to a receptor promoting internalization into target cells through receptor-mediated endocytosis (REM) is additionally bound to the hydrophilic compound.

21. The double-stranded oligo RNA molecule(s) of claim 20, wherein the ligand is selected from a group consisting of a target receptor-specific antibody, aptamer, peptide, folate, N-acetyl galactosamine (NAG), glucose, and mannose.

22. Nanoparticle(s) comprising the double-stranded oligo RNA molecule(s) of claim 7.

23. The nanoparticle(s) of claim 22, composed by mixing double-stranded oligo RNA molecules containing siRNAs comprising different sequences with each other.
Gankyrin or BMI-1 specific siRNA comprising a sense strand comprising any one sequence selected from SEQ ID NOs. 1 to 200 and an antisense strand comprising a sequence complementary thereto

24. A pharmaceutical composition comprising Gankyrin or BMI-1 specific siRNA comprising a sense strand comprising any one sequence selected from SEQ ID NOs. 1 to 200 and an antisense strand comprising a sequence complementary thereto, the double-stranded oligo RNA molecule(s) of claim 7, or nanoparticle(s) comprising said double-stranded oligo RNA molecule(s) as an active ingredient.

25. The pharmaceutical composition of claim 24, wherein it is a pharmaceutical composition for preventing or treating cancer.

26. The pharmaceutical composition of claim 25, wherein cancer is selected from a group consisting of liver cancer, gastric cancer, colon cancer, pancreatic cancer, prostate cancer, breast cancer, ovarian cancer, kidney cancer, and lung cancer.

27. The pharmaceutical composition of claim 26, wherein liver cancer is hepatocellular carcinoma (HCC).

28. Lypholized formulations comprising the pharmaceutical composition of claim 24.

29. A method for preventing or treating cancer characterized by administering Gankyrin or BMI-1 specific siRNA comprising a sense strand comprising any one sequence selected from SEQ ID NOs. 1 to 200 and an antisense strand comprising a sequence complementary thereto, the double-stranded oligo RNA molecule(s) of claim 7, or the nanoparticle(s) comprising said double-stranded oligo RNA molecule(s), to an individual requiring such treatment or prevention of cancer.

30. The method for preventing or treating cancer of claim 29, wherein cancer is selected from a group consisting of liver cancer, gastric cancer, colon cancer, pancreatic cancer, prostate cancer, breast cancer, ovarian cancer, kidney cancer, and lung cancer.

31. The method for preventing or treating cancer of claim 30, wherein liver cancer is hepatocellular carcinoma (HCC).


SEQUENCE LISTING (209 sequences)

SEQ ID NO: 1 - RNA, 19 nt, Artificial
  Gankyrin
cuguugagauuguucuacu

SEQ ID NO: 2 - RNA, 19 nt, Artificial
  Gankyrin
cuguugagauuguucuacu

SEQ ID NO: 3 - RNA, 19 nt, Artificial
  Gankyrin
cggcuguuuugacuggcgu

SEQ ID NO: 4 - RNA, 19 nt, Artificial
  Gankyrin
ggccgggaugagauuguaa

SEQ ID NO: 5 - RNA, 19 nt, Artificial
  Gankyrin
gugucuaaccuaauggucu

SEQ ID NO: 6 - RNA, 19 nt, Artificial
  Gankyrin
guggccuggguuuaauacu

SEQ ID NO: 7 - RNA, 19 nt, Artificial
  Gankyrin
cgcugucauguuacuggaa

SEQ ID NO: 8 - RNA, 19 nt, Artificial
  Gankyrin
cgcgcgacaaguaguugcu

SEQ ID NO: 9 - RNA, 19 nt, Artificial
  Gankyrin
gcugggacagcgaaaugga

SEQ ID NO: 10 - RNA, 19 nt, Artificial
  Gankyrin
guuacuuguucgaagcuua

SEQ ID NO: 11 - RNA, 19 nt, Artificial
  Gankyrin
gaaacagaacagcuccaau

SEQ ID NO: 12 - RNA, 19 nt, Artificial
  Gankyrin
ccuucugggaaaaggugcu

SEQ ID NO: 13 - RNA, 19 nt, Artificial
  Gankyrin
ccagauguuucuaugugga

SEQ ID NO: 14 - RNA, 19 nt, Artificial
  Gankyrin
ccgggaugagauuguaaaa

SEQ ID NO: 15 - RNA, 19 nt, Artificial
  Gankyrin
gagaguggaagaagcaaaa

SEQ ID NO: 16 - RNA, 19 nt, Artificial
  Gankyrin
agcccuucugggaaaaggu

SEQ ID NO: 17 - RNA, 19 nt, Artificial
  Gankyrin
aggugcucaagugaaugcu

SEQ ID NO: 18 - RNA, 19 nt, Artificial
  Gankyrin
aguuggauggugugcucua

SEQ ID NO: 19 - RNA, 19 nt, Artificial
  Gankyrin
guuaaacagcuuggauuua

SEQ ID NO: 20 - RNA, 19 nt, Artificial
  Gankyrin
gagaguauucuggccgaua

SEQ ID NO: 21 - RNA, 19 nt, Artificial
  Gankyrin
guggaagguuaaacagcuu

SEQ ID NO: 22 - RNA, 19 nt, Artificial
  Gankyrin
cuaauucuguggcuguugu

SEQ ID NO: 23 - RNA, 19 nt, Artificial
  Gankyrin
gaguauucuggccgauaaa

SEQ ID NO: 24 - RNA, 19 nt, Artificial
  Gankyrin
cccaaggagcaaguauuua

SEQ ID NO: 25 - RNA, 19 nt, Artificial
  Gankyrin
cccucccauguaccuuaua

SEQ ID NO: 26 - RNA, 19 nt, Artificial
  Gankyrin
ggauggugugcucuaaaau

SEQ ID NO: 27 - RNA, 19 nt, Artificial
  Gankyrin
uucugccagauguuucuau

SEQ ID NO: 28 - RNA, 19 nt, Artificial
  Gankyrin
gcucgcgcgacaaguaguu

SEQ ID NO: 29 - RNA, 19 nt, Artificial
  Gankyrin
ggugugugucuaaccuaau

SEQ ID NO: 30 - RNA, 19 nt, Artificial
  Gankyrin
uggauucuguaauguuccu

SEQ ID NO: 31 - RNA, 19 nt, Artificial
  Gankyrin
gaaugauaaagacgaugca

SEQ ID NO: 32 - RNA, 19 nt, Artificial
  Gankyrin
gcucaagugaaugcuguca

SEQ ID NO: 33 - RNA, 19 nt, Artificial
  Gankyrin
cgaaaaacaggcaugagau

SEQ ID NO: 34 - RNA, 19 nt, Artificial
  Gankyrin
gagauuguucuacuguugu

SEQ ID NO: 35 - RNA, 19 nt, Artificial
  Gankyrin
gcaacaagcuaguuguucu

SEQ ID NO: 36 - RNA, 19 nt, Artificial
  Gankyrin
cugucauguuacuggaagg

SEQ ID NO: 37 - RNA, 19 nt, Artificial
  Gankyrin
agaugcuaaggaccauuau

SEQ ID NO: 38 - RNA, 19 nt, Artificial
  Gankyrin
cagguuggucuccucuuca

SEQ ID NO: 39 - RNA, 19 nt, Artificial
  Gankyrin
gcuuacagcuuguuuucca

SEQ ID NO: 40 - RNA, 19 nt, Artificial
  Gankyrin
cugaguuacuuguucgaag

SEQ ID NO: 41 - RNA, 19 nt, Artificial
  Gankyrin
acuuggagugccagugaau

SEQ ID NO: 42 - RNA, 19 nt, Artificial
  Gankyrin
agcccauauaccuauguau

SEQ ID NO: 43 - RNA, 19 nt, Artificial
  Gankyrin
ccauuaugaggcuacagca

SEQ ID NO: 44 - RNA, 19 nt, Artificial
  Gankyrin
gguggaagguuaaacagcu

SEQ ID NO: 45 - RNA, 19 nt, Artificial
  Gankyrin
caucuaugaaugaugaagu

SEQ ID NO: 46 - RNA, 19 nt, Artificial
  Gankyrin
guguccuacaaacuaaugu

SEQ ID NO: 47 - RNA, 19 nt, Artificial
  Gankyrin
gugcacaagacaucaucua

SEQ ID NO: 48 - RNA, 19 nt, Artificial
  Gankyrin
guucuacuguugucguaua

SEQ ID NO: 49 - RNA, 19 nt, Artificial
  Gankyrin
guuggauggugugcucuaa

SEQ ID NO: 50 - RNA, 19 nt, Artificial
  Gankyrin
caggacagcagaacugcau

SEQ ID NO: 51 - RNA, 19 nt, Artificial
  Gankyrin
aacaauagcccauauaccu

SEQ ID NO: 52 - RNA, 19 nt, Artificial
  Gankyrin
caggccuacgccaaacguu

SEQ ID NO: 53 - RNA, 19 nt, Artificial
  Gankyrin
cuggguuuaauacucaaga

SEQ ID NO: 54 - RNA, 19 nt, Artificial
  Gankyrin
cgaagcuuacagcuuguuu

SEQ ID NO: 55 - RNA, 19 nt, Artificial
  Gankyrin
gugucauccuguauugaaa

SEQ ID NO: 56 - RNA, 19 nt, Artificial
  Gankyrin
agacgaugcagguuggucu

SEQ ID NO: 57 - RNA, 19 nt, Artificial
  Gankyrin
uucuauguggauucuguaa

SEQ ID NO: 58 - RNA, 19 nt, Artificial
  Gankyrin
cuccaauagcaacaagcua

SEQ ID NO: 59 - RNA, 19 nt, Artificial
  Gankyrin
gcuacuagaacugaccagg

SEQ ID NO: 60 - RNA, 19 nt, Artificial
  Gankyrin
ccucccauguaccuuauau

SEQ ID NO: 61 - RNA, 19 nt, Artificial
  Gankyrin
guuccuccauacaguuaaa

SEQ ID NO: 62 - RNA, 19 nt, Artificial
  Gankyrin
gugugugucuaaccuaaug

SEQ ID NO: 63 - RNA, 19 nt, Artificial
  Gankyrin
uguaauguuccuccauaca

SEQ ID NO: 64 - RNA, 19 nt, Artificial
  Gankyrin
guccuacaaacuaauguau

SEQ ID NO: 65 - RNA, 19 nt, Artificial
  Gankyrin
gaauaacuguugagauugu

SEQ ID NO: 66 - RNA, 19 nt, Artificial
  Gankyrin
guuuuugauggguuguuua

SEQ ID NO: 67 - RNA, 19 nt, Artificial
  Gankyrin
cagugaaugauaaagacga

SEQ ID NO: 68 - RNA, 19 nt, Artificial
  Gankyrin
guauucuggccgauaaauc

SEQ ID NO: 69 - RNA, 19 nt, Artificial
  Gankyrin
gcucuaacggcuguuuuga

SEQ ID NO: 70 - RNA, 19 nt, Artificial
  Gankyrin
guugcugggacagcgaaau

SEQ ID NO: 71 - RNA, 19 nt, Artificial
  Gankyrin
gauaaaucccuggcuacua

SEQ ID NO: 72 - RNA, 19 nt, Artificial
  Gankyrin
gccgggaugagauuguaaa

SEQ ID NO: 73 - RNA, 19 nt, Artificial
  Gankyrin
ggcuguacucccuuacauu

SEQ ID NO: 74 - RNA, 19 nt, Artificial
  Gankyrin
gucccaaggagcaaguauu

SEQ ID NO: 75 - RNA, 19 nt, Artificial
  Gankyrin
gagaaugguggaagguuaa

SEQ ID NO: 76 - RNA, 19 nt, Artificial
  Gankyrin
gguuaaacagcuuggauuu

SEQ ID NO: 77 - RNA, 19 nt, Artificial
  Gankyrin
cccaguguccuacaaacua

SEQ ID NO: 78 - RNA, 19 nt, Artificial
  Gankyrin
ccaguguccuacaaacuaa

SEQ ID NO: 79 - RNA, 19 nt, Artificial
  Gankyrin
ccuccauacaguuaaaaca

SEQ ID NO: 80 - RNA, 19 nt, Artificial
  Gankyrin
cgccaaacguuucuguuuu

SEQ ID NO: 81 - RNA, 19 nt, Artificial
  Gankyrin
accuaauggucugcaaccu

SEQ ID NO: 82 - RNA, 19 nt, Artificial
  Gankyrin
ucaagagaaugguggaagg

SEQ ID NO: 83 - RNA, 19 nt, Artificial
  Gankyrin
auccagaugcuaaggacca

SEQ ID NO: 84 - RNA, 19 nt, Artificial
  Gankyrin
cacucaggccuacgccaaa

SEQ ID NO: 85 - RNA, 19 nt, Artificial
  Gankyrin
ucacucaggccuacgccaa

SEQ ID NO: 86 - RNA, 19 nt, Artificial
  Gankyrin
cuguugucguauauucuuc

SEQ ID NO: 87 - RNA, 19 nt, Artificial
  Gankyrin
gggugugugucuaaccuaa

SEQ ID NO: 88 - RNA, 19 nt, Artificial
  Gankyrin
cgauaaaucccuggcuacu

SEQ ID NO: 89 - RNA, 19 nt, Artificial
  Gankyrin
gucaaucaaaauggcugua

SEQ ID NO: 90 - RNA, 19 nt, Artificial
  Gankyrin
ggcaugagaucgcugucau

SEQ ID NO: 91 - RNA, 19 nt, Artificial
  Gankyrin
gggcuaauccagaugcuaa

SEQ ID NO: 92 - RNA, 19 nt, Artificial
  Gankyrin
ccagaugcuaaggaccauu

SEQ ID NO: 93 - RNA, 19 nt, Artificial
  Gankyrin
gccuggguuuaauacucaa

SEQ ID NO: 94 - RNA, 19 nt, Artificial
  Gankyrin
ggguuuaauacucaagaga

SEQ ID NO: 95 - RNA, 19 nt, Artificial
  Gankyrin
cccucucugaaacagaaca

SEQ ID NO: 96 - RNA, 19 nt, Artificial
  Gankyrin
ccaauagcaacaagcuagu

SEQ ID NO: 97 - RNA, 19 nt, Artificial
  Gankyrin
guauguuguguuguugucc

SEQ ID NO: 98 - RNA, 19 nt, Artificial
  Gankyrin
cgaugcagguuggucuccu

SEQ ID NO: 99 - RNA, 19 nt, Artificial
  Gankyrin
aggaaguuuuaaaguaccu

SEQ ID NO: 100 - RNA, 19 nt, Artificial
  Gankyrin
ggcuguuuugacuggcgua

SEQ ID NO: 101 - RNA, 19 nt, Artificial
  BMI1
guguguucaucacccauca

SEQ ID NO: 102 - RNA, 19 nt, Artificial
  BMI1
gaaaguuucucagaaguaa

SEQ ID NO: 103 - RNA, 19 nt, Artificial
  BMI1
ugucuacauuccuucugua

SEQ ID NO: 104 - RNA, 19 nt, Artificial
  BMI1
gaauucuuugaccagaaca

SEQ ID NO: 105 - RNA, 19 nt, Artificial
  BMI1
cauugaugccacaaccaua

SEQ ID NO: 106 - RNA, 19 nt, Artificial
  BMI1
gaaauucaaccaacggaaa

SEQ ID NO: 107 - RNA, 19 nt, Artificial
  BMI1
cacaagaccagaccacuac

SEQ ID NO: 108 - RNA, 19 nt, Artificial
  BMI1
cagagagauggacugacaa

SEQ ID NO: 109 - RNA, 19 nt, Artificial
  BMI1
ggguacuucauugaugcca

SEQ ID NO: 110 - RNA, 19 nt, Artificial
  BMI1
ccagaccacuacugaauau

SEQ ID NO: 111 - RNA, 19 nt, Artificial
  BMI1
gagcuucuacagguauuuu

SEQ ID NO: 112 - RNA, 19 nt, Artificial
  BMI1
gucuacauuccuucuguaa

SEQ ID NO: 113 - RNA, 19 nt, Artificial
  BMI1
ccuggagaccagcaaguau

SEQ ID NO: 114 - RNA, 19 nt, Artificial
  BMI1
cuuuuucucuguguuagga

SEQ ID NO: 115 - RNA, 19 nt, Artificial
  BMI1
gucacugugaauaacgauu

SEQ ID NO: 116 - RNA, 19 nt, Artificial
  BMI1
gucgaacuugguguguguu

SEQ ID NO: 117 - RNA, 19 nt, Artificial
  BMI1
cagaguucgaccuacuugu

SEQ ID NO: 118 - RNA, 19 nt, Artificial
  BMI1
gacugacaaaugcuggaga

SEQ ID NO: 119 - RNA, 19 nt, Artificial
  BMI1
ccagauugaugucauguau

SEQ ID NO: 120 - RNA, 19 nt, Artificial
  BMI1
cuccaagauauuguauaca

SEQ ID NO: 121 - RNA, 19 nt, Artificial
  BMI1
cagggcuuuucaaaaauga

SEQ ID NO: 122 - RNA, 19 nt, Artificial
  BMI1
guuauuugugaggguguuu

SEQ ID NO: 123 - RNA, 19 nt, Artificial
  BMI1
cugguugauaccugagacu

SEQ ID NO: 124 - RNA, 19 nt, Artificial
  BMI1
cgagaaucaagaucacuga

SEQ ID NO: 125 - RNA, 19 nt, Artificial
  BMI1
ccacuacugaauauaaggu

SEQ ID NO: 126 - RNA, 19 nt, Artificial
  BMI1
gucagauaaaacucuccaa

SEQ ID NO: 127 - RNA, 19 nt, Artificial
  BMI1
ccaacggaaagaauaugca

SEQ ID NO: 128 - RNA, 19 nt, Artificial
  BMI1
caaccaacggaaagaauau

SEQ ID NO: 129 - RNA, 19 nt, Artificial
  BMI1
ggucagauaaaacucucca

SEQ ID NO: 130 - RNA, 19 nt, Artificial
  BMI1
gacauaagcauugggccau

SEQ ID NO: 131 - RNA, 19 nt, Artificial
  BMI1
guacucugcaguggacaua

SEQ ID NO: 132 - RNA, 19 nt, Artificial
  BMI1
gagcaagcauguugaauuu

SEQ ID NO: 133 - RNA, 19 nt, Artificial
  BMI1
gcuuggcucgcauucauuu

SEQ ID NO: 134 - RNA, 19 nt, Artificial
  BMI1
gacugugaugcacuuaaga

SEQ ID NO: 135 - RNA, 19 nt, Artificial
  BMI1
gguccacuuccauugaaau

SEQ ID NO: 136 - RNA, 19 nt, Artificial
  BMI1
cgaccuacuuguaaaagaa

SEQ ID NO: 137 - RNA, 19 nt, Artificial
  BMI1
cucacauuuccaguacuau

SEQ ID NO: 138 - RNA, 19 nt, Artificial
  BMI1
ccagcagguugcuaaaaga

SEQ ID NO: 139 - RNA, 19 nt, Artificial
  BMI1
acaagaccagaccacuacu

SEQ ID NO: 140 - RNA, 19 nt, Artificial
  BMI1
acaugugacuaucguccaa

SEQ ID NO: 141 - RNA, 19 nt, Artificial
  BMI1
aguacucugcaguggacau

SEQ ID NO: 142 - RNA, 19 nt, Artificial
  BMI1
gugguauagcaguaauuuu

SEQ ID NO: 143 - RNA, 19 nt, Artificial
  BMI1
gagaaggaaugguccacuu

SEQ ID NO: 144 - RNA, 19 nt, Artificial
  BMI1
cuguagaaaacaagugcuu

SEQ ID NO: 145 - RNA, 19 nt, Artificial
  BMI1
guaagaaucagauggcauu

SEQ ID NO: 146 - RNA, 19 nt, Artificial
  BMI1
gccaauagaccucgaaaau

SEQ ID NO: 147 - RNA, 19 nt, Artificial
  BMI1
cggguacuaccguuuauuu

SEQ ID NO: 148 - RNA, 19 nt, Artificial
  BMI1
ggugguauagcaguaauuu

SEQ ID NO: 149 - RNA, 19 nt, Artificial
  BMI1
uagagcaagcauguugaau

SEQ ID NO: 150 - RNA, 19 nt, Artificial
  BMI1
cauuaugcuuguuguacaa

SEQ ID NO: 151 - RNA, 19 nt, Artificial
  BMI1
caccaaucuucuuuugcca

SEQ ID NO: 152 - RNA, 19 nt, Artificial
  BMI1
guguguguucaucacccau

SEQ ID NO: 153 - RNA, 19 nt, Artificial
  BMI1
gccacaaccauaauagaau

SEQ ID NO: 154 - RNA, 19 nt, Artificial
  BMI1
cagcaaguauuguccuauu

SEQ ID NO: 155 - RNA, 19 nt, Artificial
  BMI1
guaugaggaggaaccuuua

SEQ ID NO: 156 - RNA, 19 nt, Artificial
  BMI1
ccucgaaaaucaucaguaa

SEQ ID NO: 157 - RNA, 19 nt, Artificial
  BMI1
gguucgaccuuugcagaua

SEQ ID NO: 158 - RNA, 19 nt, Artificial
  BMI1
gcaauuggcacaucuuucu

SEQ ID NO: 159 - RNA, 19 nt, Artificial
  BMI1
cccauuguaaguguuguuu

SEQ ID NO: 160 - RNA, 19 nt, Artificial
  BMI1
ucuauguagccaugucacu

SEQ ID NO: 161 - RNA, 19 nt, Artificial
  BMI1
ugcuuuggucgaacuuggu

SEQ ID NO: 162 - RNA, 19 nt, Artificial
  BMI1
cacaaccauaauagaaugu

SEQ ID NO: 163 - RNA, 19 nt, Artificial
  BMI1
cugugaauaacgauuucuu

SEQ ID NO: 164 - RNA, 19 nt, Artificial
  BMI1
guauuguccuauuugugau

SEQ ID NO: 165 - RNA, 19 nt, Artificial
  BMI1
cugcagcucgcuucaagau

SEQ ID NO: 166 - RNA, 19 nt, Artificial
  BMI1
cagauuggaucggaaagua

SEQ ID NO: 167 - RNA, 19 nt, Artificial
  BMI1
cagcgguaaccaccaaucu

SEQ ID NO: 168 - RNA, 19 nt, Artificial
  BMI1
cugacaaaugcuggagaac

SEQ ID NO: 169 - RNA, 19 nt, Artificial
  BMI1
cgaacaacgagaaucaaga

SEQ ID NO: 170 - RNA, 19 nt, Artificial
  BMI1
cauguaugaggaggaaccu

SEQ ID NO: 171 - RNA, 19 nt, Artificial
  BMI1
cuaauggauauugccuaca

SEQ ID NO: 172 - RNA, 19 nt, Artificial
  BMI1
gguugauaccugagacugu

SEQ ID NO: 173 - RNA, 19 nt, Artificial
  BMI1
gacauaacaggaaacagua

SEQ ID NO: 174 - RNA, 19 nt, Artificial
  BMI1
gagccuugcuuaccagcaa

SEQ ID NO: 175 - RNA, 19 nt, Artificial
  BMI1
ccuucucugcuaugucuga

SEQ ID NO: 176 - RNA, 19 nt, Artificial
  BMI1
ggucgaacuuggugugugu

SEQ ID NO: 177 - RNA, 19 nt, Artificial
  BMI1
cgaacuugguguguguuca

SEQ ID NO: 178 - RNA, 19 nt, Artificial
  BMI1
gucugcaaaagaagcacaa

SEQ ID NO: 179 - RNA, 19 nt, Artificial
  BMI1
caguacuaugaauggaacc

SEQ ID NO: 180 - RNA, 19 nt, Artificial
  BMI1
cagauggcauuaugcuugu

SEQ ID NO: 181 - RNA, 19 nt, Artificial
  BMI1
gcucgcauucauuuucugc

SEQ ID NO: 182 - RNA, 19 nt, Artificial
  BMI1
cccgcagaauaaaaccgau

SEQ ID NO: 183 - RNA, 19 nt, Artificial
  BMI1
agauggacuacaugugaua

SEQ ID NO: 184 - RNA, 19 nt, Artificial
  BMI1
ucugcaaaagaagcacaau

SEQ ID NO: 185 - RNA, 19 nt, Artificial
  BMI1
cuguaaaacguguauuguu

SEQ ID NO: 186 - RNA, 19 nt, Artificial
  BMI1
gguauaugacauaacagga

SEQ ID NO: 187 - RNA, 19 nt, Artificial
  BMI1
ggaauaugccuucucugcu

SEQ ID NO: 188 - RNA, 19 nt, Artificial
  BMI1
cugccaauggcucuaauga

SEQ ID NO: 189 - RNA, 19 nt, Artificial
  BMI1
cagcagguugcuaaaagaa

SEQ ID NO: 190 - RNA, 19 nt, Artificial
  BMI1
gauggacuacaugugauac

SEQ ID NO: 191 - RNA, 19 nt, Artificial
  BMI1
uaguaugagaggcagagau

SEQ ID NO: 192 - RNA, 19 nt, Artificial
  BMI1
uucauugaugccacaacca

SEQ ID NO: 193 - RNA, 19 nt, Artificial
  BMI1
accagcaaguauuguccua

SEQ ID NO: 194 - RNA, 19 nt, Artificial
  BMI1
agaacuggaaagugacucu

SEQ ID NO: 195 - RNA, 19 nt, Artificial
  BMI1
acuaucguccaauuugcuu

SEQ ID NO: 196 - RNA, 19 nt, Artificial
  BMI1
ucuguuccauuagaagcaa

SEQ ID NO: 197 - RNA, 19 nt, Artificial
  BMI1
guaaaauggacauaccuaa

SEQ ID NO: 198 - RNA, 19 nt, Artificial
  BMI1
gcugcucuuuccgggauuu

SEQ ID NO: 199 - RNA, 19 nt, Artificial
  BMI1
gaacagauuggaucggaaa

SEQ ID NO: 200 - RNA, 19 nt, Artificial
  BMI1
caugugacuaucguccaau

SEQ ID NO: 201 - RNA, 19 nt, Artificial
  siCONT
cuuacgcugaguacuucga

SEQ ID NO: 202 - DNA, 20 bp, Artificial
  GAPDH-F
ggtgaaggtcggagtcaacg

SEQ ID NO: 203 - DNA, 25 bp, Artificial
  GAPDH-R
accatgtagttgaggtcaatgaagg

SEQ ID NO: 204 - DNA, 20 bp, Artificial
  Gankyrin-F
agcagccaagggtaacttga

SEQ ID NO: 205 - DNA, 20 bp, Artificial
  Gankyrin-R
cacttgcaggggtgtctttt

SEQ ID NO: 206 - DNA, 20 bp, Artificial
  BMI1-F
tcatccttctgctgatgctg

SEQ ID NO: 207 - DNA, 20 bp, Artificial
  BMI1-R
ccgatccaatctgttctggt

SEQ ID NO: 208 - RNA, 19 nt, Artificial
  Gank_ref
gggcagcagccaaggguaa

SEQ ID NO: 209 - DNA, 19 bp, Artificial
  BMI_ref
cgtgtattgttcgttacct